(19) United States
(12) Patent Application Publication
(10) Pub. No.: US 2006/0204514 A1
(43) Pub. Date: Sep. 14, 2006

(54) Expression of hydrophobic proteins

(75) Inventors: Elizabeth Ann Webb, Eltham, Victoria (AU); Peter Schoofs, Greenvale Victoria (AU)

Correspondence Address: STERNE, KESSLER, GOLDSTEIN & FOX PLLC, 1100 NEW YORK AVENUE, N.W., WASHINGTON, DC, 20005, US

(73) Assignees: CSL Limited, Parkville, Victoria (AU); The Council of the Queensland Institute of Medical Research, Herston, Queensland (AU)

(21) Appl. No.: 10/521,010

(22) PCT Filed: Jul. 14, 2003
(86) PCT No.: PCT/AU03/00910

§ 371 Date: Mar. 6, 2006

(30) Foreign Application Priority Data
Jul. 12, 2002 (AU) 2002950183

(51) Int. Cl. A61K 39/02; A61K 48/00; C12N 15/74; C12N 1/21

(52) U.S. Cl. 424/190.1; 435/69.3; 435/252.33; 435/488; 424/93.2

(57) ABSTRACT

Methods are disclosed for the design of non-native (i.e. heterologous) polypeptides comprising a proportion of hydrophobic amino acids which have an increased probability of being efficiently expressed in an expression system such as a bacterial host (e.g. E. coli). The methods involve identifying one or more hydrophobic peptide sequences within a polypeptide of interest, and arranging or re-locating at least one of the hydrophobic peptide sequences within said polypeptide so as to generate a candidate polypeptide with reduced amplitude in hydrophobicity and/or length of any hydrophobic region(s). Such methods are particularly useful for designing polyepitope polypeptides, and specific examples of such are described for Epstein-Barr virus (EBV), hepatitis C virus (HCV) and human immunodeficiency virus (HIV).

### FIELD OF THE INVENTION

This invention relates to the expression of non-native (ie heterologous) polypeptides which comprise a proportion of hydrophobic amino acids in an expression system such as a bacterial host (eg E. coli). In particular, the invention provides a method for designing polyepitope polypeptides with an increased probability of being efficiently expressed (ie in amounts detectable by SDS-PAGE). One particular application of the invention relates to the production of a polyepitope polypeptide comprising cytotoxic T-cell lymphocyte (CTL) epitopes from Epstein-Barr virus (EBV) for use in a polyepitope vaccine capable of eliciting a CTL immune response for the prevention of diseases associated with EBV (eg infectious mononucleosis (IM) and nasopharyngeal carcinoma (NPC)). Other particular applications relate to the production of polyepitope polypeptides suitable for use in polyepitope vaccines for preventing and/or treating hepatitis C virus (HCV) and human immunodeficiency virus (HIV).

### BACKGROUND OF THE INVENTION

In order to maximise production of recombinant polypeptides in a bacterial host (eg E. coli), a number of parameters can be considered including factors affecting transcription (eg promoter choice, etc) and factors affecting translation mechanisms such as minimising the use of rare codons. However, these are unlikely to have an impact on the production of recombinant polypeptides comprising stretches of hydrophobic amino acids, which have traditionally proven difficult to produce in recombinant bacterial expression systems. Indeed, in the case of polypeptides comprising transmembrane sequences, the removal of these hydrophobic sequences generally improves yields of the recombinant molecule (Frace et al, 1999; Hobman et al, 1994; Polte et al, 1991; EMBL website-protein toxicity: www.embl-heidelberg.de/ExternalInfo).

The most likely reason for problems occurring in the production of foreign polypeptides possessing regions of hydrophobicity (particularly those with non-native sequences such as fusion proteins), is the post-translational association of nascent polypeptides with chaperone proteins such as E. coli groEL. GroEL is involved in the refolding process of polypeptides emerging from the ribosome and proteins will recycle through the chaperone system until the correct conformation is achieved or the protein is targeted for degradation. GroEL is known to bind hydrophobic amino acids and part of the refolding process is essentially to bury these hydrophobic sequences within the interior of the protein (Fisher and Yuan, 1994; Zahn and Pluckthun, 1994; Hayer-Hartl et al, 1994; Richarme and Kohiyama, 1994; Hendrick and Hartl, 1995; Lin et al, 1995).

Polyepitope or “polytope™” constructs (ie polypeptides comprising a tandem array of epitopes which may be contiguous or otherwise spaced apart by short intervening amino acid sequences of, for example, 1 to 5 amino acids in length), would be expected to be inherently unable to internalise any hydrophobic regions as they are not naturally-occurring sequences and lack the folding capabilities inbuilt in naturally-occurring proteins. Hence polypeptides which consist of non-native sequences, particularly those with a high proportion of hydrophobic amino acids, are likely to be sequestered in the chaperone folding pathway and ultimately targeted for degradation if a certain degree of conformational stability cannot be achieved.

Polyepitope vaccines typically comprise one or more polypeptides each made up of a tandem array of CTL epitopes. These CTL epitopes, particularly those of the HLA A2 type, often comprise predominantly hydrophobic amino acids and since HLA A2 is represented in over 40%o of the human population it is mandatory that these epitopes be included in any effective polyepitope vaccine formulation.

Examples of polyepitope vaccines are described in Australian Patent No. 736336, the entire disclosure of which is to be regarded as being incorporated herein by reference. In this patent, vaccines are described which comprise a synthetic or recombinant polypeptide, or a recombinant vaccinia virus or DNA vaccine encoding same, wherein the synthetic or recombinant polypeptide typically comprises a tandem array of CTL epitopes (eg a tandem array of 2 to 10 CTL epitopes) wherein at least two of the CTL epitopes are contiguous or spaced apart by intervening sequences in which the intervening sequences do not comprise any substantial lengths of naturally occurring flanking sequences of the epitopes. Particularly described in the prior patent are vaccines comprising a polyepitope vaccinia virus encoding a polyepitope polypeptide comprising nine CTL epitopes (each CTL epitope being of 9 to 10 amino acids in length) from EBV. Standard chromium release assays conducted with this virus in a panel of target cells expressing the HLA alleles for restriction of each epitope and using autologous CTL clones specific for each epitope as effector cells, showed that each epitope could be efficiently processed from the polyepitope polypeptide since, in all cases, the CTL clones recognised and killed the HLA matched target cell infected with the polyepitope vaccinia virus, but did not kill any of the negative controls (ie TK-vaccinia).

Further examples of polyepitope vaccines are described in International patent specification WO 01/47541, the entire disclosure of which is to be regarded as being incorporated herein by reference. In this specification, vaccines are described which comprise multiple HLA epitopes wherein the multiple HLA epitopes have been sorted so as to minimise the number of “junctional epitopes” (ie epitopes inadvertently created by the juxtaposition of two other epitopes) and wherein flanking amino acid residues are introduced wherever junctional epitopes are unavoidable.

Polyepitope vaccines may include a large number of CTL epitopes. (eg 10 or more) so that the HLA diversity of the target population is covered. It has therefore been contemplated by the present applicants to produce an EBV polyepitope vaccine which includes EBV epitopes restricted by HLA A2, A3, A11, A23, A24, B7, B8, B27, B35, B44, B46, B57, B58, B60 and B62, so as to provide protection against EBV in over 90% of the human population. This would involve the incorporation of about 26 EBV CTL epitopes into a polyepitope polypeptide. For the reasons given above, it was expected that such a polypeptide would contain hydrophobic regions and that expression in a suitable host could be highly problematical.

The work leading to the present invention was aimed at elucidating a method or procedure for overcoming the difficulties of expressing non-native polypeptides which comprise a proportion of hydrophobic amino acids (eg polyepitope polypeptides) in a bacterial host such as E. coli. The present applicants have, as a result of that work, identified a novel method for designing candidate polyepitope polypeptides with an increased probability of being efficiently expressed in a bacterial host and/or yielding a purified polyepitope polypeptide which is soluble in aqueous solutions. The identification of this method arose out of a recognition of a need for individual epitopes to be arranged in a non-random way within a polyepitope polypeptide so that regions of hydrophobicity are distributed more evenly throughout the molecule rather than clustered in one or more particular regions. The novel method therefore involves identifying one or more hydrophobic peptide sequences within a polypeptide and arranging or re-locating at least one of the hydrophobic peptide sequence(s), so as to; (a) reduce or minimise amplitude (ie peaks) in hydrophobicity across the length of the polypeptide, and/or (b) reduce or minimise the total length of any hydrophobic region(s) within the polypeptide. To assist in the utility of this method, the present applicants have also identified an algorithm which permits any number of epitopes to be readily ordered into a polyepitope polypeptide sequence lacking or having reduced regions of relative high hydrophobicity.

### SUMMARY OF THE INVENTION

In a first aspect, the present invention provides a method for designing a candidate polypeptide for expression in a prokaryotic or eukaryotic host, said method comprising,

identifying one or more hydrophobic peptide sequences within a polypeptide of interest, and

arranging or re-locating at least one of said hydrophobic peptide sequences within said polypeptide so as to generate said candidate polypeptide with reduced amplitude in hydrophobicity and/or length of any hydrophobic region(s).

Preferably, the polypeptide of interest is non-native to the intended host. Since the most preferred host is E. coli; most preferably the polypeptide is non-native to E. coli

The polypeptide of interest will preferably be a non-natural polypeptide or even a theoretical non-natural polypeptide (ie a polypeptide yet to be synthesised or expressed) comprising a plurality of amino acid sequences of interest some of which may be hydrophobic or suspected to be hydrophobic, and which has been found not to be, or is suspected not to be, efficiently expressed in said host. For such a polypeptide of interest, the method of the first aspect provides the possibility of identifying one or more hydrophobic peptide sequences, if any, within the polypeptide of interest and arranging or re-locating at least one of the hydrophobic peptide sequence(s) so as to generate a candidate polypeptide with reduced amplitude in hydrophobicity and/or length of any hydrophobic region(s), and therefore an increased probability of being efficiently expressed in a suitable host.

Preferably, the polypeptide of interest may be a synthesised or theoretical polyepitope polypeptide comprising a tandem array of epitopes of interest (eg CTL epitopes, which, as is mentioned above, often predominantly comprise hydrophobic amino acids). For such a polypeptide of interest, the method of the first aspect permits the design of candidate polyepitope polypeptides comprising a large number of epitopes of interest (eg 5 to 100 or more) with an increased probability of being efficiently expressed in a suitable host, by enabling the possibility of identifying one or more hydrophobic epitopes and arranging or re-locating at least one of the hydrophobic epitope(s), so as to generate a candidate polyepitope polypeptide with reduced amplitude in hydrophobicity and/or length of any hydrophobic region(s).

It has been found that the method of the first aspect is best applied to the design of a candidate polyepitope polypeptide in a manner which identifies and ranks the relative hydrophobicity of each of the selected epitopes (nb The epitopes of interest may be a range of epitopes from a single pathogen (eg EBV) selected to provide a polyepitope polypeptide that covers the HLA diversity of the target population. The epitopes of interest may also be one or more epitopes from a range of pathogens or the epitopes may be derived from a non-microbial source such as a tumour cell for treating or preventing cancer.), groups the ranked epitopes into three groups of substantially equivalent numbers, based upon the identified relative hydrophobicity (ie so as to produce the groups, Group 1=most hydrophobic, Group 2=middle hydrophobicity, and Group 3=least hydrophobic and “residual” epitopes where the total number of epitopes (N) is not wholly divisible by 3), and then arranges the epitopes into triplets where the triplets contain an epitope from each group (ie three linked epitopes; epitope 1-epitope 2-epitope 3) and arranged into a candidate polyepitope polypeptide having the formula, Triplet 1-Triplet 2- . . . -Triplet N/3, as follows:,

| | Epitope 1 | Epitope 2 | Epitope 3 |
|---|---|---|---|
| Triplet 1 (N-terminal) | Most | Most | Most |
| | hydrophilic of | hydrophobic of | hydrophilic |
| | Group 2 | Group 1 | of Group 3 |
| Triplet 2 | 2^{nd} most | 2^{nd} most | 2^{nd} most |
| | hydrophilic | hydrophobic | hydrophilic |
| | of Group 2 | of Group 1 | of Group 3 |
| Triplet N/3 (C- | Most | Most | Most |
| terminal) | hydrophobic of | hydrophilic of | hydrophobic of |
| | Group 2 | Group 1 | Group 3 |

(Any “leftover” epitope(s) (ie least hydrophilic epitope(s) of Group 3) may be added to the C-terminal of Triplet N/3, or otherwise may be located within the candidate polyepitope polypeptide sequence so as to reduce any local peaks of hydrophobicity.)

Between the epitope triplets, or between any or all of the epitopes within a triplet, there may be intervening sequences (preferably short sequences of 1 to 10 amino acids) which may optionally be hydrophilic (eg lysine-lysine) so as to reduce any local peaks of hydrophobicity. Preferably, the epitopes within a triplet are contiguous.

Other simple methods for arranging the epitope(s) so as to minimise extremes in hydrophobicity in a polyepitope polypeptide will be readily apparent to persons skilled in the art, and are to be considered as forming part of the present invention. For example, in one variation of the above method, and referring again to Table 1, epitope 1 would instead be selected using the stated criteria for epitope 2, epitope 2 would instead be selected using the stated criteria for epitope 3, and epitope 3 would instead be selected using the stated criteria for epitope 1. In another variation, the epitopes would be selected from four groups of ranked epitopes and consequently arranged into sets of 4 epitopes.

As mentioned above, the method of the first aspect generates a candidate polypeptide with reduced amplitude in hydrophobicity and/or length of any hydrophobic region(s). In the context of applying the method to a natural polypeptide, “reduced amplitude in hydrophobicity” is to be understood to mean that any peaks of hydrophobicity of the candidate polypeptide (ie as may be calculated/measured using Pinsoft 2 from Mimotopes Pty Ltd, Melbourne, Australia) is reduced relative to the natural polypeptide, and that “reduced length of any hydrophobic region(s)” is to be understood to mean that the length of amino acid sequence of any hydrophobic region(s) in the candidate polypeptide is/are reduced relative to the natural polypeptide. In the context of applying the method to a non-natural polypeptide (including a theoretical non-natural polypeptide), “reduced amplitude in hydrophobicity” is similarly to be understood to mean that any peaks of hydrophobicity of the candidate polypeptide (ie as may be calculated/measured using Pinsoft 2 from Mimotopes Pty Ltd) is reduced relative to the non-natural polypeptide, and that “reduced length of any hydrophobic region(s)” is to be understood to mean that the length of amino acid sequence of any hydrophobic region(s) in the candidate polypeptide is/are reduced relative to the non-natural polypeptide. In the context of applying the method to the more specific non-natural polypeptide example of a polyepitope polypeptide, “reduced amplitude in hydrophobicity” is to be understood to mean that any peaks of hydrophobicity of the candidate polypeptide (ie as may be calculated using the mathematical expression described below) is reduced relative to most of the possible random arrangements of the epitopes comprising the polyepitope polypeptide, and that “reduced length of any hydrophobic region(s)” is to be understood to mean that the length of amino acid sequence of any hydrophobic region(s) in the candidate polypeptide is/are reduced relative to most of the possible random arrangements of the epitopes within the polyepitope polypeptide.

Once a candidate polypeptide has been designed in accordance with the method of the first aspect, a polynucleotide encoding the candidate polypeptide may be synthesised according to any of the methods well known to persons skilled in the art. The encoding polynucleotide may be incorporated into, for example, vectors such as viral vectors (eg vaccinia to provide a recombinant polyepitope viral vaccine) or expression vectors such as those suitable for expression in a suitable host.

Thus, in a second aspect, the present invention provides a method of expressing a polypeptide in a suitable host, said method comprising,

designing a polypeptide in accordance with the method of the first aspect,

introducing a polynucleotide encoding said polypeptide into said host, such that said host is capable of expressing said polypeptide, and

culturing said host under conditions suitable for expression of said polypeptide.

The expressed polypeptide may be isolated by, for example, lysing the host cell and purifying the polypeptide from the produced cell lysate.

The polynucleotide introduced into the host cell may encode the polypeptide in the form of a fusion of the polypeptide with a suitable carrier protein. Alternatively, the polypeptide could be expressed and subsequently linked to or otherwise associated with a suitable carrier protein. Suitable carrier proteins are well known to persons skilled in the art and include β-galactosidase, glutathione S-transferase and the gp350 structural protein from EBV or a fragment thereof. The carrier protein may comprise additional useful epitopes. Further increases in expression benefits provided by ordering may be conferred by the carrier protein.

In a third aspect, the present invention provides a polypeptide designed in accordance with the method of the first aspect.

If desired, the polypeptide of the third aspect may be in the form of a fusion of the polypeptide with a suitable carrier protein.

In a fourth aspect, the present invention provides a polyepitope polypeptide designed in accordance with the method of the first aspect.

If desired, the polypeptide of the fourth aspect may be in the form of a fusion of the polypeptide with a suitable carrier protein.

In a fifth aspect, the present invention provides a polyepitope polypeptide comprising N epitopes, wherein N is any integer (preferably an integer in the range of 5 to 100, and more preferably, 10 to 35), said polyepitope polypeptide having the formula;
Triplet 1-Triplet 2- . . . -Triplet N/3,
wherein each of said triplets comprises three linked epitopes selected by,

identifying and ranking the relative hydrophobicity of each of the N epitopes,

grouping the ranked N epitopes into three groups of substantially equivalent numbers, based upon the identified relative hydrophobicity of the N epitopes, to produce a first group (ie Group 1) comprising the most hydrophobic epitopes, a second group (ie Group 2) comprising the epitopes having a middle level of hydrophobicity, and a third group (ie Group 3) comprising the least hydrophobic epitopes, and

selecting the epitopes for each of said triplets according to the following table:

| | Epitope 1 | Epitope 2 | Epitope 3 |
|---|---|---|---|
| Triplet 1 (N-terminal) | Most | Most | Most |
| | hydrophilic of | hydrophobic of | hydrophilic of |
| | Group 2 | Group 1 | Group 3 |
| Triplet 2 | 2^{nd} most | 2^{nd} most | 2^{nd} most |
| | hydrophilic | hydrophobic | hydrophilic |
| | of Group 2 | of Group 1 | of Group 3 |
| Triplet N/3 (C- | Most | Most | Most |
| terminal) | hydrophobic of | hydrophilic of | hydrophobic of |
| | Group 2 | Group 1 | Group 3 |

Preferably, the first, second and third groups comprise identical numbers of epitopes. Where N is an integer not wholly divisible by 3 (ie an integer other than, for example, 6, 9, 12, 15, and 18), then the residual epitopes are preferably included within the third group.

At the end of the step of selecting epitopes for each of said triplets, if there is/are any leftover epitope(s) (ie least hydrophilic epitope(s) of Group 3), then this/these may be added to the C-terminal of Triplet N/3, or otherwise may be located within the candidate polyepitope polypeptide sequence so as to reduce any local peaks of hydrophobicity.

Between the epitope triplets, or between any or all of the epitopes within a triplet, there may be intervening sequences (preferably short sequences of 1 to 10 amino acids) which may optionally be hydrophilic (eg lysine-lysine) so as to reduce any local peaks of hydrophobicity, or otherwise avoid the creation of junctional epitopes. Preferably, the epitopes within a triplet are contiguous.

If desired, the polyepitope polypeptide of the fifth aspect may be in the form of a fusion of the polyepitope polypeptide with a suitable carrier protein.

In a sixth aspect, the present invention provides a polypeptide vaccine comprising a polyepitope polypeptide according to the fourth or fifth aspect and a pharmaceutically acceptable carrier and/or adjuvant.

In a seventh aspect, the present invention provides a polyepitope polypeptide comprising an amino acid sequence substantially corresponding to an amino acid sequence selected from the group consisting of:

(SEQ ID NO:1)

FLRGRAYGL-PYLFWLAAI-HRCQAIRKK-RRIYDLIEL-
VQPPQLTLQV-GLCTLVAML-RLRAEAQVK-IEDPPFNSL-
YLLEMLWRL-GQGGSPTAM-AVLLHEESM-IALYLQQNWWTL-
RAKFKQLL-SSCSSCPLSKI-TYGPVFMCL-QAKWRLQTL-
RPPIFIRRL-VSFIEFVGW-YPLHEQHGM-VEITPYKPT-CLGGLLTMV-
EENLLDFVRF-TYSAGIVQI-LLDFVRFMGV-EGGVGWRHW,

(SEQ ID NO:2)

FLRGRAYGL-PYLFWLAAI-HRCQAIRKK-RRIYDLIEL-GLCTLVAML-
RLRAEAQVK-IEDPPFNSL-TYSAGIVQI-LLDFVRFMGV-
EGGVGWRHW-IALYLQQNWWTL-RAKFKQLL-SSCSSCPLSKI-
TYGPVFMCL-QAKWRLQTL-RPPIFIRRL-VSFIEFVGW-YPLHEQHGM-
VEITPYKPTW-CLGGLLTMV-EENLLDFVRF-YLLEMLWRL-
GQGGSPTAM-AVLLHEESM-VQPPQLTLQV,

(SEQ ID NO:3)

SSCSSCPLSKI-HRCQAIRKK-CLGGLLTMV-LTAGFLIFL-
RLRAEAQVK-IEDPPFNSL-LLSAWILTA-RRRWRRLTV-PYLFWLAAI-
YLLEMLWRL-GQGGSPTAM-VMSNTLLSAW-ALLVLYSFA-RAKFKQLL-
IALYLQQNW-TYGPVFMCL-QAKWRLQTL-YLQQNWWTL-YPLHEQHGM-
CPLSKILL,

(SEQ ID NO:4)

IPIVAIVALV-RLRPGGKKK-ILKEPVHGV-PLVKLWYQL-
RPGGKKKYKL-KYKLKHIVW-TWETWWTEYW-EIKDTKEAL-
KRWIILGLNK-KLWVTVYYGV-KIEELRQHL-MTNNPPIPV-
VTLWQRPLV-WASRELERF-LLWKGEGAV-YTAFTIPSI-
IYQEPFKNLK-SLYNTVATL-AIIRILQQL-AIFQSSMTK-
VIYQYMDDL-LVGPTPVNI-TPQDLNTML-YLAWVPAHK-ALVEICTEM-
TLNAWVKVV,
and

(SEQ ID NO:5)

LLFNILGGWV-KTSERSQPR-FLLLADARV-LLFLLLADA-
RLGVRATRK-GVAGALVAFK-LPGCSFSIF-RMYVGGVEHR-
VAGALVAFK-DLMGYIPLV-LIFCHSKKK-ILAGYGAGV-HMWNFISGI-
QLFTFSPRR-VGIYLLPNR-FWAKHMWNF-YLVTRHADV-LSAFSLHSY-
WMNRLIAFA-YLLPRRGPRL-YLVAYQATV-RLIVFPDLGV-
TLGFGAYMSK-IPFYGKAI-VLVGGVLAA-CTCCSSDLY.

In an eighth aspect, the present invention provides a polyepitope vaccine comprising a polyepitope polypeptide according to the seventh aspect and a pharmaceutically acceptable carrier and/or adjuvant.

### DETAILED DESCRIPTION OF THE INVENTION

The present applicants have identified novel methods for designing a candidate polyepitope polypeptide, with an increased probability of being efficiently expressed in a prokaryotic or eukaryotic host (ie in amounts detectable by SDS-PAGE). The method involves identifying one or more hydrophobic epitope(s) and arranging or re-locating at least one of the hydrophobic epitope(s) so as to generate a candidate polyepitope polypeptide with reduced amplitude in hydrophobicity and/or length of any hydrophobic region(s).

An algorithm to calculate hydrophobicity values of amino acid sequences and subsequently arrange sequences to; (a) reduce or minimise amplitude in hydrophobicity, and/or (b) reduce or minimise the length of hydrophobic sequences, was generated and initially applied to 26 CTL epitope sequences from EBV. This resulted in the design of two initial candidate polyepitope polypeptides (designated PT26A and PT26B, described hereinafter), one of which proved to be efficiently expressed in E. coli. The expressed polyepitope polypeptide shows promise as the basis of an EBV vaccine for prevention or treatment of infectious mononucleosis and/or EBV-related cancers such as Burkitts lymphoma, Hodgkin's disease, non-Hodgkin's lymphoma, nasopharyngeal carcinoma, gastric adenocarcinoma, lymphomas associated with immunosupression, lymphoepithelioma-like carcinomas, and immunodeficiency-related leiomyosarcoma.

While looking for an explanation as to why the expression capabilities of E. coli for the two similar candidate polyepitope polypeptides were different, summations of hydrophobicity values (designated Hydrophobic Index (HI) values) were calculated for different numbers of epitopes over the length of the candidate polyepitope polypeptides to identify local areas of hydrophobicity. Summation over 3 and 4 epitopes showed that there were regions in the non-expressed polypeptide where the HI value was higher than in the expressed polypeptide. This information enabled the identification of a threshold HI value, such that polypeptide sequences which comprised a region with an H value in excess of the threshold value, could be predicted as being less likely of being efficiently expressed in a bacterial host.

Thus, in a preferred embodiment of the methods of the present invention for designing candidate polypeptides, the methods involve initially calculating hydrophobicity values and arranging peptide sequences to; (a) reduce or minimise amplitude in hydrophobicity, and (b) reduce or minimise the length of hydrophobic sequences; and then “fine-tuning”, if necessary, by calculating the HI values over different peptide sequence groups, thus providing numerical values for comparison and prediction of the likelihood of a candidate polypeptide sequence being efficiently expressed in a bacterial host. So, in applying this preferred embodiment to the design of a candidate polyepitope polypeptide, the method involves:
(i) Calculating the hydrophobic value for each epitope using a suitable algorithm (eg Fauschere and Pliska, 1983 contained within the software package “Pinsoft 2” from Mimotopes Pty Ltd).
(ii) Ranking the set of epitopes in order of decreasing hydrophobicity.
(iii) Dividing the rank ordered set of epitopes into a number of equal groups. (eg three equal groups wherein group 1 =most hydrophobic, group 2 =middle hydrophobicity and group 3 =least hydrophobic (most hydrophilic)), and including any residual epitopes in the most hydrophilic group.
(iv) Creating sets (eg triplets) of epitopes by taking, in for example a case where the epitopes have been divided into three groups, the most hydrophilic epitope of group 2 (ie last in group 2), then the most hydrophobic epitope (ie number 1 in group 1) and lastly the most hydrophilic epitope (ie last in group 3) until all epitopes in groups 1 and 2 have been used (nb “Leftover” epitopes are handled as set out in step (ix) below).
(v) Arranging the sets of epitopes (eg triplets) into a sequence in the order in which they were produced (eg Triplet 1-Triplet 2-Triplet 3-etc).
(vi) Plotting the hydrophobicity of the arranged polyepitope polypeptide sequence using a suitable algorithm (eg Fauschere and Pliska, 1983, or Hopp and Woods, 1981).
(vii) If necessary, reducing hydrophobic amplitude by re-locating sets of epitopes (eg triplets) from areas of low hydrophobicity into areas of high peak hydrophobicity and/or by re-locating individual hydrophobic (ie group 1) epitopes from areas of peak hydrophobicity into areas of low hydrophobicity.
(viii) Re-calculating the hydrophobicity plots and continuing, if necessary, to shuffle sets of epitopes (eg triplets) as in step (vii) above to generate a final sequence arrangement.
(ix) Placing any leftover epitopes (eg least hydrophilic epitopes of group 3) at the C-terminal of the final sequence arrangement or other location so as to further reduce local peaks in hydrophobicity (ie by inserting them adjacent to epitopes of peak hydrophobicity).
(x) Placing any affinity tags (usually hydrophilic, eg a hexa-histidine sequence) at either the N— or C-terminal of the final polyepitope polypeptide sequence or at the C-terminal if the final polyepitope polypeptide sequence is to be expressed as a fusion protein.

The HI values may be calculated by using the mathematical expression:
e=m+n−1
HIₘ=Σxₑ
e=m
(where m=group number, n=group size, e=integer (epitope) number, x=epitope hydrophobicity value).

Preferably, the HI values are calculated using this mathematical expression when n=3 and n=4. In the examples provided hereinafter, this calculation predicted that to be able to express linked, random, short amino acid sequences in E. coli in SDS-PAGE detectable amounts, the hydrophobic index over groups of three epitope sequences would need to be less than 1.8 (HI₃<1.8) and/or that over groups of four epitope sequences, the hydrophobic index would need to be less than 2.5 when x was calculated using Pinsoft 2 (Mimotopes Pty Ltd) and specifying the N-terminus as N-acetyl and the C-terminus as carboxy amide. Different cut-off values will be obtained with different hydrophobicity algorithms.

It will be readily appreciated that the calculation of HI values in this manner, would be useful for predicting whether a natural, non-bacterial polypeptide or a derivative thereof may be efficiently expressed in E. coli The present invention therefore further extends to a method of predicting efficient expression of a polypeptide in a suitable host (eg a bacterial host), involving calculating HI values in accordance with the mathematical expression:
e=m+n−1
HIₘ=Σxₑ
e=m
(where m=group number, n=group size, e=integer (epitope) number, x =epitope hydophobicity value).

The methods of the present invention permit the design of candidate polyepitope polypeptides comprising a large number of epitopes of interest (eg 5 to 100 or more) with an increased probability of being efficiently expressed in a suitable host (eg a bacterial host). The epitopes of interest may be a range of epitopes from a single pathogen selected to provide a polyepitope polypeptide that covers the HLA diversity of the target population, or the epitopes of interest may be one or more epitopes from a range of pathogens or tumour antigens. As is evident from the above, one particular application of the methods of the present invention is to the design of candidate polyepitope polypeptides comprising 26 EBV CTL epitopes for use in a vaccine to provide protection against EBV in over 90% of the human population, or for treating diseases associated with EBV such as NPC. Other particular applications of the methods of the present invention relate to the design of candidate polyepitope polypeptides suitable for use in polyepitope vaccines for preventing and/or treating HCV and HIV. Another particular application of the methods of the present invention is to the design of candidate polyepitope polypeptides comprising CTL epitopes from cytomegalovirus (CMV), for use in a vaccine to prevent or treat CMV-causative diseases.

A candidate polypeptide designed in accordance with the methods of the present invention may be expressed by firstly synthesising a polynucleotide encoding the candidate polypeptide according to any of the methods well known to persons skilled in the art, and then by introducing the polynucleotide into a suitable host. Typically, this will be achieved by cloning the polynucleotide into an expression vector and then introducing the expression vector into said host by any of the transformation methods well known to persons skilled in the art. Expression from the expression vector may result in the polypeptide being expressed as a fusion protein comprising the polypeptide and a suitable carrier protein (eg β-galactosidase, glutathione S-transferase or the gp350 structural protein from EBV or a fragment thereof). Alternatively, the polypeptide may be expressed by the host cell, and following isolation of the polypeptide, the polypeptide may be linked to or otherwise associated with a suitable carrier protein. The carrier protein may also confer additional useful properties (ie the carrier protein may comprise useful epitopes or sequences to enhance solubility, further enhance purification procedures, facilitate association with an adjuvant or to which an immune response is desirable).

It is further contemplated that candidate polypeptides designed in accordance with the methods of the present invention may be expressed in prokaryotic expression systems other than E. coli. Typical alternative systems include B. subilis, Salmonella sp., Streptococcus sp., Lactobacillus sp., and Streptomyces sp.

It is also contemplated that candidate polypeptides designed in accordance with the methods of the present invention may be readily expressed in whole cell lysates and non-bacterial host cells as well, and accordingly such alternative expression methods for candidate polypeptides are to be considered as forming part of the present invention. In particular, the present invention is to be considered as extending to a method of expressing a polypeptide in a non-bacterial host cell such as a mammalian cell (eg a CHO cell or COS cell line), a yeast cell (eg Saccharomyces cerevisiae) or insect cell (eg SF9 cell line), wherein the method comprises designing a polypeptide in accordance with the method of the first aspect, introducing a polynucleotide encoding the polypeptide into the host cell such that the host cell is capable of expressing the polypeptide, and culturing the host cell under conditions suitable for expression of the polypeptide. The expressed polypeptide may be isolated from the host cell culture by lysing the cells and purifying the polypeptide from the produced cell lysate, or alternatively, the polypeptide could be expressed with a suitable secretion signal such that the polypeptide is secreted into the culture medium (from where it may be purified). Designing a polyepitope polypeptide in accordance with the methods of the present invention may also overcome non-secretion problems which are sometimes experienced when a hydrophobic polypeptide is expressed with a foreign secretion signal.

Where the expressed polypeptide is of pharmacological or veterinary significance, the polypeptide may be formulated into a pharmaceutical or veterinary composition. Generally, such compositions will comprise a pharmaceutically acceptable or veterinary acceptable carrier, and may include other substances and excipients as may be required.

Polyepitope polypeptides may be formulated into vaccine compositions. Generally, such compositions will comprise a pharmaceutically acceptable or veterinary acceptable carrier and may include adjuvants (eg an ISCOM™ adjuvant, DEAE, polysaccharides, saponins, liposomes and virus-like particles), and other substances and excipients as may be required. The vaccine compositions may also include helper epitopes/CD4 epitopes or B-cell epitopes. The vaccine compositions may be adapted for administration to a subject by, for example, intramuscular injection, nasal administration via an aerosol spray, or oral administration. Preferably, the vaccine compositions are ISCOM™ adjuvant compositions.

Polyepitope polypeptides may also be administered to a subject in the form of a viral vaccine (eg a recombinant polyepitope vaccinia or adenovirus) or DNA vaccine. Thus, in a further aspect, the present invention provides a polynucleotide vaccine comprising a polynucleotide encoding a polypeptide designed in accordance with the method of the first aspect, and a pharmaceutically acceptable carrier and/or adjuvant.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “substantially corresponding” as used herein in relation to an amino acid sequence is intended to encompass the exact amino acid sequence as well as minor variations which do not result in a substantial decrease in the biological activity of the amino acid sequence (eg variations which do not diminish the ability of an epitope to provoke a CTL immune response). These variations may include one or more conservative amino acid substitutions. The conservative amino acid substitutions envisaged are: G, A, V, I, L, M; D, E, N, Q; S, C, T; K, R, H; and P, Nα-alkylamino acids.

Any discussion of documents, acts, materials, devices, articles or the like which has been included in the present specification is solely for the purpose of providing a context for the present invention. It is therefore, not to-be taken as an admission that any or all of these matters form part of the prior art base or were common general knowledge in the art relevant to the present invention as it existed in Australia or elsewhere before the filing or priority date of the present specification.

The invention will hereinafter be further described by way of the following non-limiting examples and accompanying figures.

### BRIEF DESCRIPTION OF THE ACCOMPANYING FIGURES

FIG. 1 provides the epitope configuration and amino acid sequences for EBV-polyepitope polypeptides, PT26A and PT26B. Numbers above epitopes represent hydrophobicity values for each epitope calculated with Pinsoft 2 software, specifying an acetyl N-terminal and an amide C-terminal.

FIG. 2 provides hydrophobicity plots of PT26A and PT26B. Hydrophobicity values of a moving nine amino acid window are derived using the algorithm of Fauchere and Pliska, 1983.

FIG. 3 provides the amino acid sequence of the fusion of residues 21 to 447 of EBV gp350 to PT26A (A) or to PT26B (B).

FIG. 4 shows Coomassie stained SDS-PAGE gels showing the time course of expression following induction with IPTG (+IPTG or I) of: (A) PT26A (at approximately 30 kDa), (B) gp350/PT26A (at approximately 80 kDa), and (C) PT26B expression. Arrows indicate the location of recombinant protein.

FIG. 5 provides ELISPOT assay results of CTL responses to the five HLA A2 epitopes contained within the polyepitope polypeptides, PT26A and PT26B, under two formulation conditions: (A) 10 μg gp350-PT26A, (B) 10 μg gp350-PT26B, and (C) positive control peptide mix containing a mixture of each of the 5 A2 epitopes contained in the polypeptide polypeptide. Each of the epitopes is represented below by their first 3 amino acids. The CTL response of each mouse M1-M5 to each A2 epitope is presented as a bar indicating the number of IFN-γ spots produced.

FIG. 6 provides the epitope configuration and amino acid sequences for an EBV polyepitope polypeptide, EBV-NPCa. Numbers above epitopes represent hydrophobicity values for each epitope calculated with Pinsoft 2 software, specifying an acetyl N-terminus and an amide C-terminus.

FIG. 7 provides the epitope configuration and amino acid sequences for HIV polyepitope polypeptides, HIVa and HIVb. Numbers above epitopes represent hydrophobicity values for each epitope calculated-with Pinsoft 2 software, specifying an acetyl N-terminus and an amide C-terminus. Hydrophobic Index values across 3 epitopes (n=3) are shown below.

FIG. 8 provides the epitope configuration and amino acid sequences for HCV polyepitope polypeptides, HCVa and HCVb. Numbers above epitopes represent hydrophobicity values for each epitope calculated with Pinsoft 2 software, specifying an acetyl N-terminus and an amide C-terminus. Hydrophobic Index values across 3 epitopes (n=3) are shown below.

FIGS. 9 and 10 show Coomassie stained SDS-PAGE gels and also immunoblots from an expression time course of constructs HIVa, HIVb, HCVa and HCVb. FIG. 9—Panel A: 1 hour timepoint. Lane 1) Novex SeeBlue+2 MW markers; 2) Negative vector/host control; 3) HIVa uninduced; 4) HIVa induced; 5) HIVb uninduced; 6) HIVb induced; 7) HCVa uninduced; 8) HCVa induced; 9) HCVb uninduced; 10) HCVb induced. Panel B: 2 hour timepoint. Lane 1) Negative vector/host control; 2) Novex SeeBlue+2 MW markers; 3) HIVa uninduced; 4) HIVa induced; 5) HIVb uninduced; 6) HIVb induced; 7) HCVa uninduced; 8) HCVa induced; 9) HCVb uninduced. FIG. 10—Panel A: 3 hour timepoint. Lane 1) Negative vector/host control; 2) HIVa uninduced; 3) HIVa induced; 4)HIVb uninduced; 5) HIVb induced; 6) Novex SeeBlue+2 MW markers; 7) HCVa uninduced; 8) HCVa induced; 9) HCVb uninduced; 10) HCVb induced. Panel B: Overnight timepoint. Lane 1) Negative vector/host control; 2) HIVa uninduced; 3) HIVa induced; 4)HIVb uninduced; 5) HIVb induced; 6) HCVa uninduced; 7) HCVa induced; 8) HCVb uninduced; 9) HCVb induced; 10) Novex SeeBlue+2 MW markers.

### EXAMPLE 1

### EBV Polyepitope Fusions as Vaccine Candidates

Materials and Methods

Epitope Sequences

The 26 CTL epitopes for inclusion in an EBV vaccine, the proteins from which they originate and HLA type are shown in Table 1.

TABLE 1

CTL epitopes included in the EBV polytopes

| | | |
|---|---|---|
| HLA Type | EBV Protein | Epitope |

| | | |
|---|---|---|
| A2 | LMP2 | CLGGLLTMV (SEQ ID NO:6) |
| | BMLF1 | GLCTLVAML (SEQ ID NO:7) |
| | EBNA6 | LLDFVRFMGV (SEQ ID NO:8) |
| | LMP1 | YLLEMLWRL (SEQ ID NO:9) |
| | LMP1 | YLQQNWWTL (SEQ ID NO:10) |
| A3 | EBNA3 | RLRAEAQVK (SEQ ID NO:11) |
| A11 | LMP2 | SSCSSCPLSKI (SEQ ID NO:12) |
| A23 | LMP2 | PYLFWLAAI (SEQ ID NO:13) |
| A24 | LMP2A | TYGPVFMCL (SEQ ID NO:14) |
| | EBNA4 | TYSAGIVQI (SEQ ID NO:15) |
| B7 | EBNA3 | RPPIFIRRL (SEQ ID NO:16) |
| B8 | EBNA3 | FLRGRAYGL (SEQ ID NO:17) |
| | EBNA3 | QAKWRLQTL (SEQ ID NO:18) |
| | BZLF1 | RAKFKQLL (SEQ ID NO:19) |
| B27 | EBNA4 | HRCQAIRKK (SEQ ID NO:20) |
| | EBNA6 | RRIYDLIEL (SEQ ID NO:21) |
| B35 | EBNA4 | AVLLHEESM (SEQ ID NO:22) |
| | EBNA3 | YPLHEQHGM (SEQ ID NO:23) |
| B44 | EBNA6 | EENLLDFVRF (SEQ ID NO:24) |
| | EBNA6 | EGGVGWRHW (SEQ ID NO:25) |
| | EBNA4 | VEITPYKPTW (SEQ ID NO:26) |
| B46 | EBNA3 | VQPPQLTLQV (SEQ ID NO:27) |
| B57 | LMP1 | IALYLQQNW (SEQ ID NO:28) |
| B58 | EBNA4 | VSFIEFVGW (SEQ ID NO:29) |
| B60 | LMP2 | IEDPPFNSL (SEQ ID NO:30) |
| B62 | EBNA4 | GQGGSPTAM (SEQ ID NO:31) |

Design/Ordering of Epitopes

The following method was used to generate ordered arrangements of CTL epitopes to produce a polyepitope sequence with favourable hydrophobicity characteristics:
(i) The hydrophobic value for each epitope was calculated using a suitable algorithm (ie Pinsoft 2 from Mimotopes Pty Ltd, specifying the N-terminus as N-acetyl and the C-terminus as carboxy amide).
(ii) The set of epitopes was ranked in order of decreasing hydrophobicity.
(iii) The rank ordered set of epitopes-was divided into 3 equal groups (ie group 1=most hydrophobic, group 2=middle hydrophobicity and group 3=least hydrophobic (most hydrophilic)). Residual epitopes (ie 2 epitopes left over after the set of 26 was divided by 3), were included in the most hydrophilic group.
(iv) Triplets of epitopes were created by taking the most hydrophilic of group 2 (ie last in group 2), then the most hydrophobic epitope (ie number 1 in group 1) and lastly the most hydrophilic (ie last in group 3). This was continued until all epitopes in groups 1 and 2 had been used (nb “Leftover” epitopes were added to the C-terminal end of the final sequence arrangement).
(v) The triplets were then arranged into a sequence in the order in which they were produced (ie Triplet 1-Triplet 2-Triplet 3-etc).
(vi) The hydrophobicity of this triplet arrangement was then plotted using a suitable algorithm (ie Fauschere and Pliska).
(vii) If and where necessary, relocating triplets from areas of low hydrophobicity into areas of high peak hydrophobicity in order to reduce hydrophobic amplitude.
(viii) Re-calculating the hydrophobicity plots and continuing, if necessary, to shuffle triplets as in the step (vii) above.
(ix) Any residual epitopes (ie least hydrophilic of group 3) can be placed C-terminally in the final sequence arrangement or can be used to further reduce local peaks in hydrophobicity by inserting them adjacent to epitopes of peak hydrophobicity, according to a hydrophobicity plot of the assembled triplets.
(x) Any affinity tags (usually hydrophilic, eg a hexa-histidine sequence) should be located either N— or C-terminally or preferably C-terminally if the construct is a fusion protein.
(xi) Confirmation of satisfactory H.

Using this process, EBV polyepitope configurations PT26A and PT26B were created. The example of PT26A is shown in Table 4.

Hydrophobic Index (HI) Calculations

HI values for favourable configurations (PT26A, PT26B) were calculated according to the mathematical expression:—
e=m+n−1
HIₘ=Σxₑ
e=m
(where m group number, n=group size, e=integer (epitope) number, x=epitope hydophobicity value) over values of n from 2 to 5.
Preparation of Recombinant Proteins

DNA sequences encoding the polyepitopes (PT26A and PT26B) were generated from synthetic oligonucleotides using a Splicing by Overlap Extension technique (SOE) as described by Horton et al (1995). The codon usage was optimised for E. coli expression (Wada et al 1992). The polyepitopes were tagged at the C-terminus with a hexa-histidine tag for protein purification and detection. The DNA was subcloned into pET28b (Novagen) and transformed into E. coli BL21(DE3) cells (Novagen) for expression.

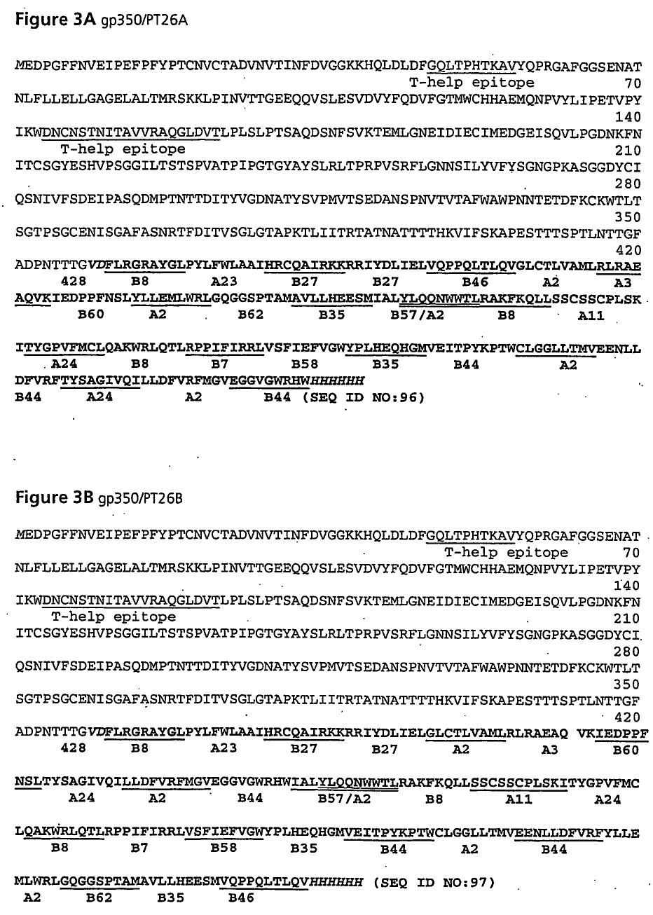

A fragment corresponding to the N-terminal region (amino acid residues 21-447) of EBV gp350 was amplified from plasmid DNA containing the full length gp350 sequence by PCR using the following oligonucleotides: 5′ AGGGATCCCATGGAAGATCCTGGTTTTTTC 3′ (forward) (SEQ ID NO:32) and 5′ TCTAGAGGTCGACACCTGTCGTTGTATTGGG 3′ (reverse) (SEQ ID NO:33). This DNA fragment was subcloned into pET28b (Novagen) containing the polyepitope insert, resulting in an in-frame fusion between gp350 and the polyepitope polypeptide. The constructs were referred to as gp350/PT26A (FIG. 3A) and gp350/PT26B (FIG. 3B).

For protein expression testing, transformed cells were grown in 50 mg/ml Kanamycin containing L broth at 37° C. until OD600 reached 2. Protein expression was induced by the addition of IPTG (0.25 mM) and cells were grown for another 3 hours. Cells were pelleted and boiled in SDS sample buffer before analysing by SDS-PAGE.

For protein purification, cells induced by IPTG were pelleted, resuspended in binding buffer (20 mM Tris-HCl pH 7.9, 0.5M NaCl, 5 mM imidazole) and then sonicated. Inclusion bodies were pelleted and washed in buffer. The proteins were solubilised overnight in binding buffer containing 8M urea and purified on a Ni⁺⁺-NTA column.

Preparation of ISCOM™ Formulations

ISCOMATRIX™-adjuvant was prepared by combining adjuvant components in a formulation vessel. Cholesterol, 1,2, dipalmitoyl phosphatidylcholine (DPPC), and ISCOPREP™ as a source of purified Quillaja saponins, were mixed in a weight ratio of 1:1:5 in the presence of the detergent Mega-10 (U.S. Pat. No. 5,679,354) at a concentration of 2%. The detergent was removed by diafiltration with PBS and the formation of ISCOMATRIX™ confirmed by negative contrast electron microscopy revealing complexes including cage-like structures typically with a diameter of 40 nm. ISCOM™-adjuvanted vaccines were prepared by mixing the EBV polyepitope antigen with preformed ISCOMATRIX™-adjuvant, which was prepared as described below: The dose strength of ISCOM-adjuvant as saponin was quantified by reverse phase HPLC assay.

ISCOM™ vaccines-were prepared by gentle mixing at 22° C. of an equal volume of 2× final dose strength ISCOMATRIX™ with an equal volume of 2× final dose strength EBV polyepitope antigen (gp350-PT26A and B). After 60 minutes, the formulation was subjected to extensive dialysis, in order to remove urea, at 4° C. into PBS buffer pH6.2 using 12,000 molecular weight cut off dialysis membrane (Cellu Sep T3, San Antonio Tex.).

Mouse Immunogenicity

Dosing

Female HLA A2 transgenic C57B1/6 mice (HDD) were bred at Queensland Institute of Medical Research (QIMR, Brisbane, Australia) and immunised at 5-7 weeks of age. Mice were housed in filter-topped cages in the PC3 animal facility at QIMR. Groups of 4 or 5 mice were dosed sub-cutaneously at the tail base with 0.1 ml formulation. This was followed by removal of spleens at day 21 for ex-vivo ELISPOT assay (below).

Mice dosed sub-cutaneously, received 10 μg ISCOM™-adjuvant (as saponin) and 10 μg EBV polyepitope antigen. For a vaccine control group, mice were dosed subcutaneously with a peptide mixture comprising free peptides (Mimotopes Pty Ltd) formulated with tetanus toxoid and Montanide ISA 720 (SEPPIC, Paris, France) as previously described (Elliot et al, 1999). Peptide control immunisations come from two groups of mice, one group immunised with GLCTLVAML(SEQ ID NO:6)/YLLEMLWRL(SEQ ID NO:9)/LLDFVRFMGV(SEQ ID NO:8) peptide mixture and the other YLQ/CLG. A mixture of all five epitopes showed some insolubility problems.

CTL Activity (ex-vivo ELLISPOT)

Ex vivo ELISPOT measures/quantitates both effector and memory CTL which secrete IFNγ. Peptide-specific IFNγ secreting cells are enumerated by an enzyme linked immuno-spot (ELISpot) assay modified from Murali-Krishna et al. Flat bottomed 96-well microtitre plates are coated overnight with 5 ug/mL of rat anti-mouse IFNγ antibody(clone RA-6A2, BD PharMingen, San Diego, Calif., USA). Coated plates are then blocked for 1 hour with 1% FBS in PBS, and then washed three times with PBS/0.05% Tween 20 (PBS-T), and incubated for 1 hour at 37° C. with medium comprising RPMI 1640, supplemented with 100 μg/ml streptomycin and 100 IU/ml penicillin, 10% FBS and 10⁻⁵ M 2-mercaptoethanol. Mouse splenocytes were then treated with red blood cell lysis buffer, washed and resuspended to 1×10⁷ cells/ml in medium, for use in the ex vivo IFNγ ELISPOT assay. Splenocytes (1×10⁶/well) are then placed in the first wells of the ELISpot-plate and serially diluted two fold. Recombinant human IL-2 (kindly provided by Cetus Corp., Emeryville, Calif., USA) is then added to the plate at a final concentration of 5 IU/well together with EBV peptide (Mimotopes Pty Ltd) at a final concentration of 100 μg/ml. Media containing IL-2 without peptide is added to control wells. The final volume in each well is 100 μl. Plates are incubated at 37° C. in 5% CO₂ for approximately 18 hours. After incubation, cells are lysed by rinsing the plates in H₂O and then washed twice in PBS-T. Biotinylated anti-mouse IFNγ antibody clone XMG1.2 (BD Phar Mingen) is diluted 1:500 (2 μg/ml final concentration) in PBS-T/5% FBS and added to all wells at 50 μl/well and incubated for 2 hours at RT. Plates are then washed in PBS-T and streptavidin-alkaline phosphatase, diluted 1:400 in PBS-T/5% FBS, is added at 50 μl per well and incubated for a further 2 hours. After washing, plates are developed by adding Sigma Fast BCIP/NBT substrate at 50 ul/well. Plates are incubated at 37° C. for approximately 20 minutes to allow colour development, and then washed with water to stop the reaction. IFNγ specific spots are counted using KS ELISPOT Reader (Zeiss).

Results

Epitope Fusions

26 EBV CTL epitopes were selected to provide >90% human population coverage for a vaccine formulation.

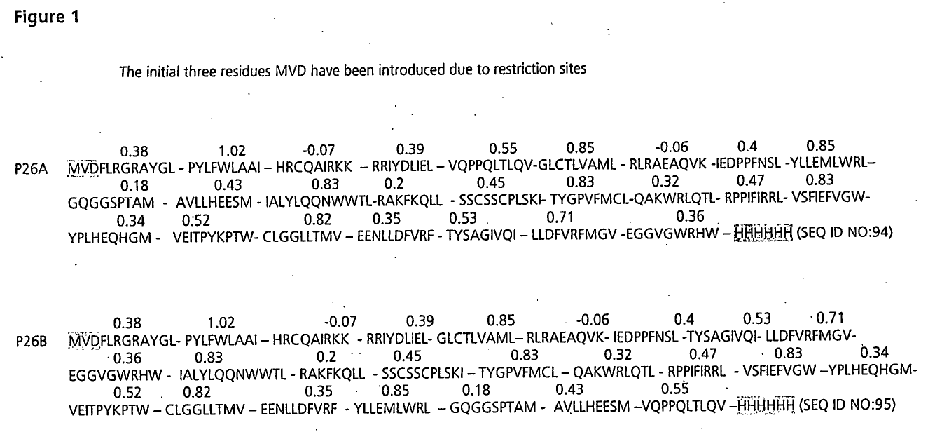

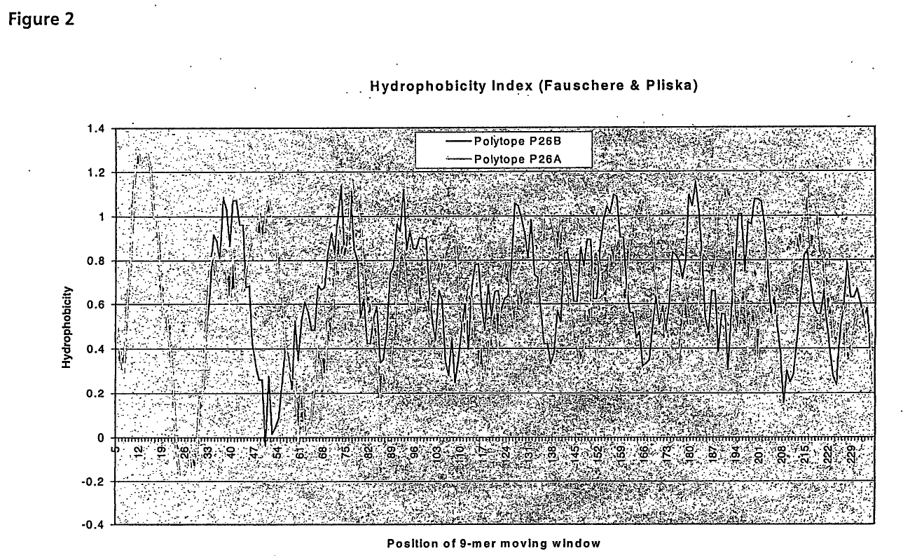

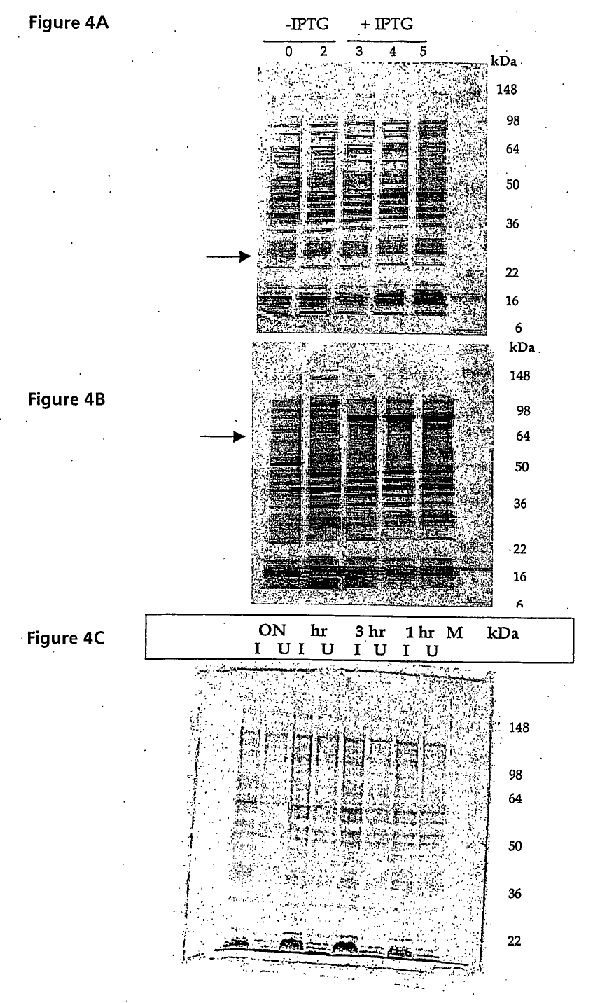

In order to link these CTL epitopes (Table 1) together and facilitate the design of a polyepitope antigen to form the basis of an EBV prophylactic vaccine, the hydrophobicity value of each epitope was calculated using Pinsoft 2 software (Table 2). Two versions of 26 epitopes were then ordered into configurations PT26A and PT26B (FIG. 1), which reduced peak hydrophobicity and hydrophobic sequence length (FIG. 2). When these constructs were cloned for expression in E. coli it was found that only one of the configurations (PT26A) was able to produce a polyepitope polypeptide (FIG. 4A). PT26B was not produced (FIG. 4C).

To identify a potential reason for this unexpected finding, local areas of high hydrophobicity were examined by summation of overlapping hydrophobicity values (Pinsoft 2, Mimotopes Pty Ltd) to provide a hydrophobicity index (I) over varying numbers of peptides in a group (n). For n=2, no correlation was apparent. However, for n=3 and n=4 (Table 3), the highest HI values for the expressed sequence (PT26A) were lower (1.79 and 2.51 respectively) than the highest values obtained for the non-expressed sequence (2.02 and 2.54 respectively). For n=5, again no significant differences were seen. This would indicate that there were local areas of slightly higher hydrophobicity in PT26B than PT26A.

The above analysis can be represented by the mathematical expression:—
e=m+n−1
HIₘ=Σxₑ
e=m
(where m group number, n=group size, e=integer (epitope) number, x=epitope hydophobicity value).

Overall, the results with n=3 and n=4 appeared to show the most distinctive differences. This would make the prediction that to be able to express linked, random, short amino acid sequences in E. coli SDS-PAGE detectable amounts, the hydrophobic index over groups of three epitope sequences would preferably need to be less than 1.8 (HI₃<1.8) and/or that over groups of four the hydrophobic index would preferably need to be less than 2.5 (HI₄<2.5), where x was calculated using Pinsoft 2 (Mimotopes Pty Ltd) and specifying the N-terminus as N-acetyl and the C-terminus as carboxy amide. Different cut-off values will be obtained with different hydrophobicity algorithms.

Table 4 shows the HI values over 3 peptides (n=3) for 15 random arrangements of the 26 EBV CTL epitopes that were generated and analysed by calculating the HI. This shows that all 15 random configurations contain multiple HI values which are all in the range predicted to prelude the production of a recombinant polypeptide in E. coli (ie HI greater than 1.8). This also shows that the arrangements made in accordance with the present methods are unlikely to be arrived at without application of the insights embodied in the present invention.

TABLE 2
Ordering process for generation of a polyepitope polypeptide for 26* EBV CTL epitopes (PT26A).
The hydrophobicity value for each epitope is calculated, then the epitopes are rank ordered, and grouped into triplets. The sequence is
assessed using a hydrophobicity plot and scored for HI value by summing the epitope hydrophobicity values over a moving 3-mer window.
If necessary, fine-tuning of the epitope order is done and the sequence reassessed. The final epitope order and amino acid sequence for the
26* EBV CTL epitopes and hexa-histidine affinity tag is shown below.

| | | Hydro- | | | | | | | |
|---|---|---|---|---|---|---|---|---|---|
| | | pho- | | | | | | | Sum |
| | | bicity | | | | | | | Hyd |
| | | (Hyd) | | | | | | | for |
| HLA | | (Pinsoft | Rank ordered | | | | Order after fine | | trip- |
| Type | EPITOPE | 2) | on Hyd | Hyd | Grouped into triplets | Hyd. | tuning | Hyd | lets |
| A2 | CLGGLLTMV | 0.82 | PYLFWLAAI | 1.02 | FLRGRAYGL | 0.38 | FLRGRAYGL | 0.38 | |
| A2 | GLCTLVAML | 0.85 | GLCTLVAML | 0.85 | PYLFWLAAI | 1.02 | PYLFWLAAI | 1.02 | |
| A2 | LLDFVRFMGV | 0.71 | YLLEMLWRL | 0.85 | HRCQAIRKK | −0.07 | HRCQAIRKK | −0.07 | 1.33 |
| A2 | YLLEMLWRL | 0.85 | IALYLQQNWWTL* | 0.83 | RRIYDLIEL | 0.39 | RRIYDLIEL | 0.39 | 1.34 |
| A2/B57 | IALYLQQNWWTL* | 0.83 | TYGPVFMCL | 0.83 | GLCTLVAML | 0.85 | VQPPQLTLQV | 0.55 | 0.87 |
| | (SEQ ID NO: 34) | | | | | | | | |
| A3 | RLRAEAQVK | −0.06 | VSFIEFVGW | 0.83 | RLRAEAQVK | −0.06 | GLCTLVAML | 0.85 | 1.79 |
| A11 | SSCSSCPLSKI | 0.45 | CLGGLLTMV | 0.82 | IEDPPFNSL | 0.40 | RLRAEAQVK | −0.06 | 1.34 |
| A23 | PYLFWLAAI | 1.02 | LLDFVRFMGV | 0.71 | YLLEMLWRL | 0.85 | IEDPPFNSL | 0.40 | 1.19 |
| A24 | TYSAGIVQI | 0.53 | VQPPQLTLQV | 0.55 | GQGGSPTAM | 0.18 | YLLEMLWRL | 0.85 | 1.19 |
| A24 | TYGPVFMCL | 0.83 | TYSAGIVQI | 0.53 | AVLLHEESM | 0.43 | GQGGSPTAM | 0.18 | 1.43 |
| B7 | RPPIFIRRL | 0.47 | VEITPYKPTW | 0.52 | IALYLQQNWWTL* | 0.83 | AVLLHEESM | 0.4 | 1.46 |
| | | | | | | | | identi- | |
| | | | | | | | | fying 3 | |
| B8 | FLRGRAYGL | 0.38 | RPPIFIRRL | 0.47 | RAKFKQLL | 0.20 | IALYLQQNWWTL* | 0.83 | 1.44 |
| B8 | QAKWRLQTL | 0.32 | SSCSSCPLSKI | 0.45 | SSCSSCPLSKI | 0.45 | RAKFKQLL | 0.20 | 1.46 |
| B8 | RAKFKQLL | 0.20 | AVLLHEESM | 0.43 | TYGPVFMCL | 0.83 | SSCSSCPLSKI | 0.45 | 1.48 |
| B27 | HRCQAIRKK | −0.07 | IEDPPFNSL | 0.40 | QAKWRLQTL | 0.32 | TYGPVFMCL | 0.83 | 1.48 |
| B27 | RRIYDLIEL | 0.39 | RRIYDLIEL | 0.39 | RPPIFIRRL | 0.47 | QAKWRLQTL | 0.32 | 1.60 |
| B35 | YPLHEQHGM | 0.34 | FLRGRAYGL | 0.38 | VSFIEFVGW | 0.83 | RPPIFIRRL | 0.47 | 1.62 |
| B35 | AVLLHEESM | 0.43 | EGGVGWRHW | 0.36 | YPLHEQHGM | 0.34 | VSFIEFVGW | 0.83 | 1.62 |
| B44 | VEITPYKPTW | 0.52 | EENLLDFVRF | 0.35 | VEITPYKPTW | 0.52 | YPLHEQHGM | 0.34 | 1.64 |
| B44 | EGGVGWRHW | 0.36 | YPLHEQHGM | 0.34 | CLGGLLTMV | 0.82 | VEITPYKPTW | 0.52 | 1.69 |
| B44 | EENLLDFVRF | 0.35 | OAKWRLQTL | 0.32 | EENLLDFVRF | 0.35 | CLGGLLTMV | 0.82 | 1.68 |
| B46 | VQPPQLTLQV | 0.55 | RAKFKQLL | 0.20 | TYSAGIVQI | 0.53 | EENLLDFVRF | 0.35 | 1.69 |
| B58 | VSFIEFVGW | 0.83 | GQGGSPTAM | 0.18 | LLDFVRFMGV | 0.71 | TYSAGIVQI | 0.53 | 1.70 |
| B60 | IEDPPFNSL | 0.40 | RLRAEAOVK | −0.06 | EGGVGWRHW | 0.36 | LLDFVRFMGV | 0.71 | 1.59 |
| B62 | GQGGSPTAM | 0.18 | HRCQAIRKK | −0.07 | VQPPQLTLQV | 0.55 | EGGVGWRHW | 0.36 | 1.60 |
| | HHHHHH | 0.04 | HHHHHH | 0.04 | HHHHHH | 0.04 | HHHHHH | 0.04 | 1.11 |
| | (SEQ ID NO: 35) | | | | | | | | |

(*The epitope IALYLQQNWWTL is comprised of two overlapping CTL epitopes IALYLQQNW and YLQQNWWTL that were combined for this study.)

TABLE 3
3mers (n = 3)

| | | | | | | | | | | | | |
|---|---|---|---|---|---|---|---|---|---|---|---|---|
| PT26A | 1.33 | 1.34 | 0.87 | 1.79 | 1.34 | 1.19 | 1.19 | 1.43 | 1.46 | 1.44 | 1.46 | 1.48 |
| PT26B | 1.33 | 1.34 | 1.17 | 1.18 | 1.19 | 0.87 | 1.64 | 1.6 | 1.9 | 1.39 | 1.48 | 1.48 |

4mers (n = 4)

| | | | | | | | | | | | | |
|---|---|---|---|---|---|---|---|---|---|---|---|---|
| PT26A | 1.72 | 1.89 | 1.72 | 1.73 | 1.74 | 2.04 | 1.37 | 1.86 | 2.29 | 1.64 | 1.91 | 2.31 |
| PT26B | 1.72 | 2.19 | 1.11 | 1.58 | 1.72 | 1.58 | 2 | 2.43 | 2.1 | 1.84 | 2.31 | 1.8 |

| 3mers (n = 3) | | | | | | | | | | | |
|---|---|---|---|---|---|---|---|---|---|---|---|
| PT26A | 1.48 | 1.6 | 1.62 | 1.62 | 1.64 | 1.69 | 1.68 | 1.69 | 1.7 | 1.59 | 1.6 |
| PT26B | 1.6 | 1.62 | 1.62 | 1.64 | 1.69 | 1.68 | 1.69 | 2.02 | 1.38 | 1.46 | 1.16 |
| 4mers (n = 4) | | | | | | | | | | | |
| PT26A | 1.8 | 2.07 | 2.45 | 1.96 | 2.16 | 2.51 | 2.03 | 2.22 | 2.41 | 1.95 | |
| PT26B | 2.07 | 2.45 | 1.96 | 2.16 | 2.51 | 2.03 | 2.54 | 2.2 | 1.81 | 2.01 | |

TABLE 4
Hydrophobic Index

3mers (n = 3)

| PT26B | 1.33 | 1.34 | 1.17 | 1.18 | 1.19 | 0.87 | 1.64 | 1.6 | 1.9 | 1.39 | 1.48 | 1.48 |
|---|---|---|---|---|---|---|---|---|---|---|---|---|
| PT26A | 1.33 | 1.34 | 0.87 | 1.79 | 1.34 | 1.19 | 1.19 | 1.43 | 1.46 | 1.44 | 1.46 | 1.48 |
| Random | | | | | | | | | | | | |
| 1 | 1.93 | 1.62 | 1.41 | 0.93 | 1 | 1.67 | 1.66 | 1.64 | 0.72 | 0.91 | 0.82 | 1.21 |
| 2 | 0.69 | 0.64 | 1.54 | 1.65 | 1.75 | 1.31 | 1.39 | 0.88 | 1.51 | 1.81 | 2.58 | 2.41 |
| 3 | 1.67 | 1.47 | 1.62 | 2 | 1.52 | 1.1 | 0.45 | 0.96 | 1.58 | 1.8 | 1.33 | 1.61 |
| 4 | 1.86 | 1.5 | 1.71 | 1.28 | 1.36 | 1.19 | 1.17 | 1.56 | 2.05 | 2.52 | 2.15 | 2.13 |
| 5 | 1.67 | 1.36 | 1.44 | 1.88 | 1.68 | 1.58 | 0.93 | 0.99 | 0.49 | 0.51 | 0.98 | 1.88 |
| 6 | 1.1 | 0.94 | 1.39 | 1.46 | 2.11 | 1.46 | 1.58 | 1.22 | 1.47 | 1.74 | 1.62 | 2.02 |
| 7 | 1.03 | 0.58 | 0.72 | 1.34 | 1.6 | 1.22 | 0.85 | 1.05 | 0.94 | 1.14 | 1.06 | 1.22 |
| 8 | 1.48 | 1.88 | 1.52 | 2.03 | 1.7 | 1.41 | 1.25 | 0.66 | 0.82 | 0.65 | 1.74 | 1.75 |
| 9 | 2.04 | 1.83 | 1.75 | 1.16 | 1.02 | 1.45 | 1.85 | 2 | 2.01 | 1.56 | 1.74 | 1.74 |
| 10 | 1.24 | 1.15 | 0.71 | 1.62 | 1.69 | 2.13 | 1.68 | 1.57 | 1.29 | 1.24 | 1.72 | 2.02 |
| 11 | 0.52 | 0.94 | 0.86 | 1.03 | 1.1 | 1.23 | 1.26 | 1.66 | 2.06 | 1.88 | 1.52 | 1.5 |
| 12 | 1.56 | 1.86 | 1.97 | 1.46 | 1.45 | 1.4 | 2.05 | 1.16 | 0.72 | 0.26 | 0.88 | 1.96 |
| 13 | 0.68 | 0.74 | 1.36 | 1.83 | 1.72 | 1.57 | 0.92 | 1.4 | 1.35 | 1.7 | 1.35 | 1.32 |
| 14 | 1.4 | 1.35 | 1.36 | 1.17 | 1.62 | 2.06 | 1.62 | 1.24 | 1.39 | 1.2 | 0.91 | 0.7 |
| 15 | 0.52 | 1.03 | 1.67 | 2 | 2.02 | 1.85 | 1.67 | 1.63 | 1.61 | 1.66 | 1.55 | 1.27 |
| | | | | | | | | | | | | # > |
| 3mers (n = 3) | | | | | | | | | | | | 1.8 |
| PT26B | 1.6 | 1.62 | 1.62 | 1.64 | 1.69 | 1.68 | 1.69 | 2.02 | 1.38 | 1.46 | 1.16 | 2 |
| PT26A | 1.48 | 1.6 | 1.62 | 1.62 | 1.64 | 1.69 | 1.68 | 1.69 | 1.7 | 1.59 | 1.6 | 0 |
| Random | | | | | | | | | | | | |
| 1 | 1.51 | 1.09 | 1.29 | 0.89 | 1.5 | 2 | 2.42 | 2.25 | 2.05 | 2.03 | 1.85 | 7 |
| 2 | 1.94 | 1.75 | 1.24 | 1.69 | 1.49 | 1.98 | 1.58 | 1.44 | 0.81 | 0.91 | 1.55 | 5 |
| 3 | 1.74 | 1.88 | 1.9 | 1.8 | 1.67 | 0.76 | 0.8 | 1.24 | 1.62 | 1.86 | 1.39 | 5 |
| 4 | 1.21 | 1.78 | 1.15 | 1.69 | 0.85 | 0.59 | 0.51 | 1.16 | 1.56 | 1.69 | 1.41 | 5 |
| 5 | 2.08 | 2.25 | 2.13 | 2.2 | 1.53 | 0.76 | 1.11 | 1.61 | 2.03 | 1.55 | 1.25 | 7 |
| 6 | 1.91 | 2.58 | 2.05 | 1.87 | 1.23 | 1.74 | 1.73 | 1.28 | 1.28 | 1.15 | 1.15 | 6 |
| 7 | 1.27 | 1.78 | 2.11 | 2.02 | 2.21 | 1.71 | 2.2 | 1.63 | 1.81 | 1.8 | 2.18 | 7 |
| 8 | 1.31 | 0.68 | 1.16 | 1.65 | 1.79 | 1.78 | 2.18 | 1.97 | 1.51 | 1.53 | 1.61 | 4 |
| 9 | 1.76 | 1.17 | 1.19 | 1.15 | 1.53 | 1.39 | 0.96 | 0.82 | 0.56 | 1.48 | 1.82 | 6 |
| 10 | 1.85 | 1.74 | 1.42 | 2.26 | 1.91 | 1.81 | 1.11 | 1.58 | 1.35 | 1.55 | 0.65 | 6 |
| 11 | 1.82 | 2.18 | 2.37 | 2.38 | 1.48 | 0.85 | 1.17 | 1.95 | 1.9 | 1.6 | 1.71 | 8 |
| 12 | 1.92 | 1.77 | 1.58 | 2.06 | 2.02 | 1.53 | 0.88 | 0.99 | 1.5 | 1.84 | 1.82 | 9 |
| 13 | 1.64 | 2 | 2.51 | 2.37 | 2.39 | 2.08 | 1.31 | 1.48 | 1.16 | 1.6 | 1.01 | 6 |
| 14 | 0.84 | 1.18 | 1.41 | 1.92 | 2.23 | 2.51 | 2.09 | 1.62 | 1.14 | 1.53 | 1.88 | 6 |
| 15 | 1.7 | 1.77 | 1.74 | 1.25 | 0.79 | 1.09 | 1.58 | 2.5 | 1.86 | 1.74 | 1.25 | 5 |

Fusions of These Polyepitopes with the N-Terminal 400 Amino acids of a Naturally-Occurring EBV Protein (gp350).

Provision of CD4 help has previously been shown to improve CTL induction (Thuy et al, 2001) and the EBV structural protein gp350 was identified as the preferred candidate to provide this property because it would provide cognate help. Hence the two polyepitopes, PT26A and PT26B, the latter of which was unable to be expressed in E. coli, were cloned onto the C-terminus of the N-terminal 400 amino acids of gp350 (FIG. 3) and expressed as the fusion protein in E. coli. Both of these fused polypeptides were well-produced, being clearly visible on a Coomassie-stained gel above the background of E. coli proteins (profile for PT26A shown in FIG. 4B).

CTL Activity

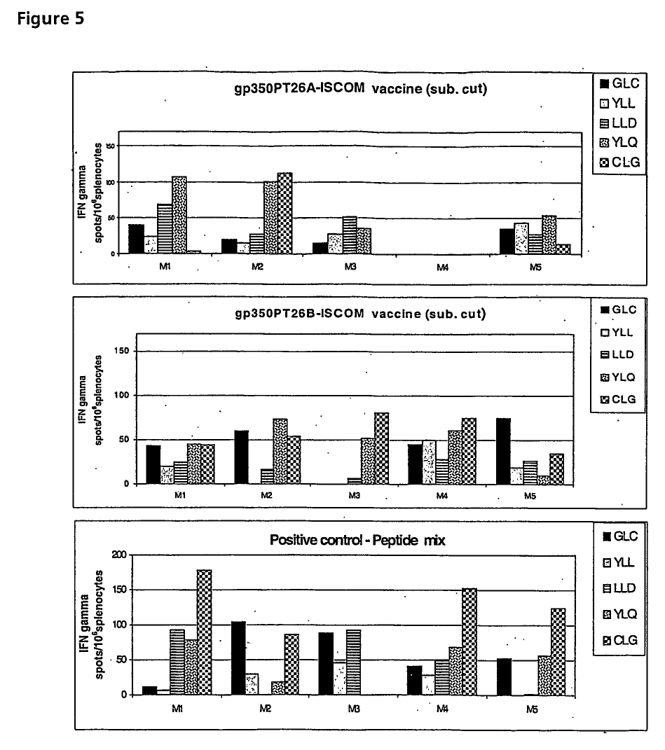

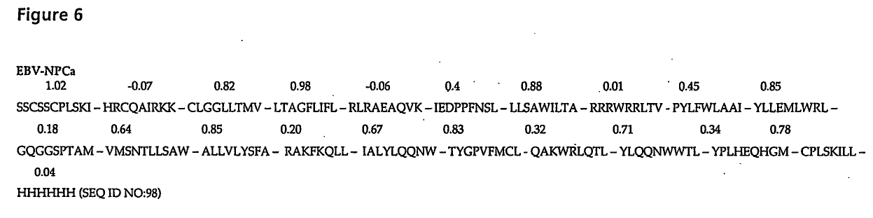

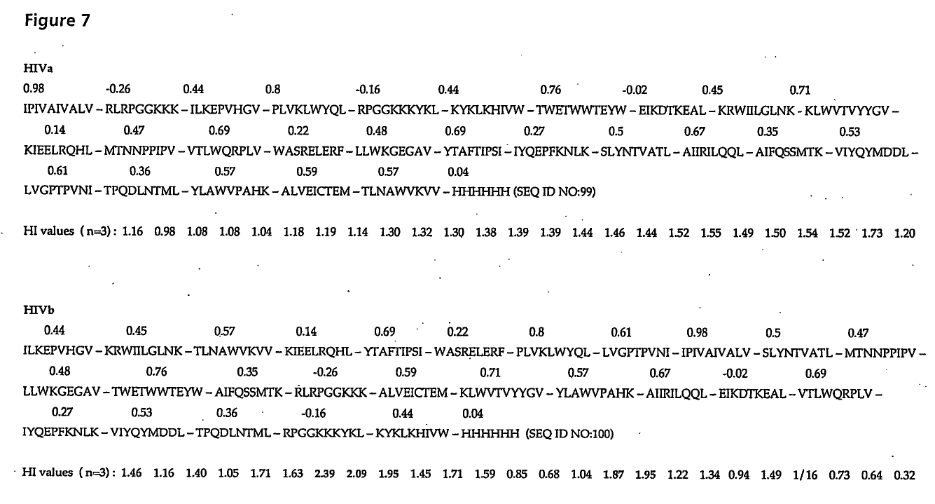

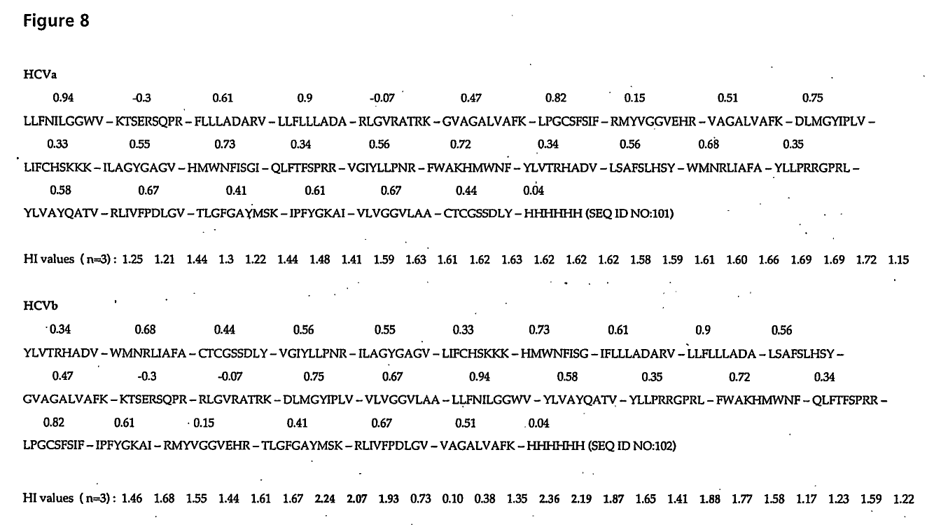

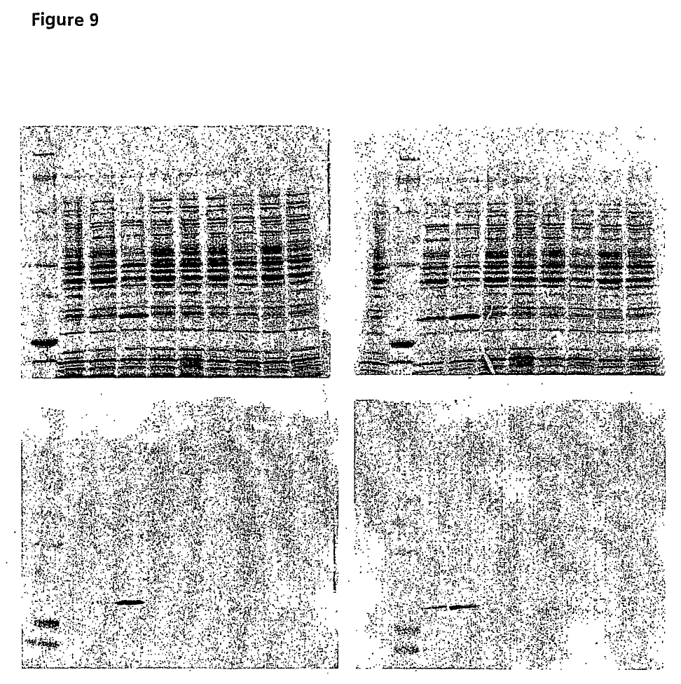

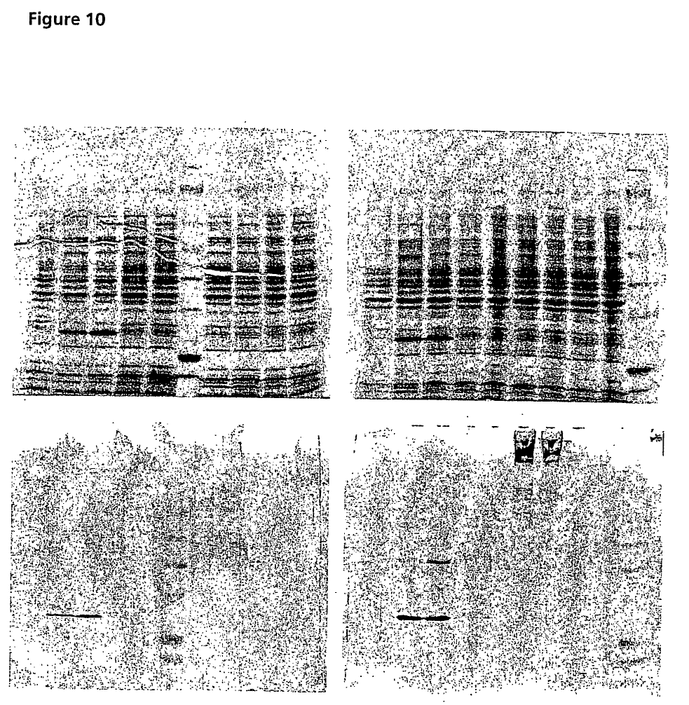

Vaccination of HLA A2 transgenic mice with either gp350-PT26A-ISCOM™ vaccine or gp350-PT26B-ISCOM™ vaccine induced a CTL immune response (IFN ELISPOT) to all five A2 epitopes (FIG. 5), thus indicating that all A2 epitopes were properly processed and presented to the immune system.

### EXAMPLE 2

### EBV Polyepitope Fusions as Nasopharyngeal Carcinoma Vaccine Candidates

Materials and Methods

Epitope Sequences

The 20 CTL epitopes for inclusion in an EBV-NPC vaccine, the proteins from which they originate and HLA type are shown in Table 5.

Design/Ordering of Epitopes

An ordered arrangement of CTL epitopes to produce a polyepitope sequence with favourable hydrophobicity characteristics was generated by the method described in Example 1 (i)-(xi).

The final shuffle involved taking YPLHEQHGM (SEQ ID NO:23) from position 3 and changing with CLGGLLTMV (SEQ ID NO:16) at position 19 to reduce a high hydrophobic index (HI) which resulted from summing epitopes 18, 19 and 20.

The ordering process for this optimised EBV-NPC polyepitope (EBV-NPCa) configuration is shown in Table 6.

Hydrophobic Index (W) Calculations

HI values for EBV-NCPa were calculated according to the mathematical expression:—
e=m+n−1
HIₘ=Σxₑ
e=m
(where m=group number, n=group size, e=integer (epitope) number, x=epitope hydophobicity value) over values of n from 2 to 5).
Generation of DNA Constructs

The amino acid sequence of the EBV-NPC polyepitope was back translated to DNA using the Dnastar Editseq software (DNASTAR Inc, Madison, Wis., USA) and codons optimised for E. coli expression. A C-terminal hexa-histidine tag was incorporated for purification and detection. The DNA encoding the polyepitope was generated from synthetic oligonudeotides using a Splicing by Overlap Extension technique (SOE) as described by Horton et al (1995). This was cloned into pET24b (Novagen) and the resulting construct sequenced to ensure that no errors were present (Big Dye Terminator Kit V3.1; Applied Biosystems). For expression purposes, DNA was transformed into E. coli BL21(DE3) cells (Novagen).

Protein Expression and Analysis

Tranformed cells were grown at 37° C. in Terrific Broth containing 50 mg/ml Kanamycin. At an OD600 of ˜2, protein expression was induced by addition of 0.5 mM IPTG and samples taken at 1 hour, 2 hours, 3 hours and overnight post-induction. Cells were pelleted, resuspended to equal densities and boiled in SDS sample buffer prior to analysing by SDS-PAGE on Novex 4-20% Tris-Glycine gels. Gels were analysed both by Coomassie Blue staining and immunoblotting. Blots were probed with Dianova anti hexa-histidine monoclonal antibody.

1. A method for designing a candidate polypeptide for expression in a suitable host, said method comprising,
identifying one or more hydrophobic peptide sequences within a polypeptide of interest, and
arranging or re-locating at least one of said hydrophobic peptide sequences within said polypeptide
so as to generate said candidate polypeptide with reduced amplitude in hydrophobicity or length of a hydrophobic region.

2. (canceled)

3. (canceled)

4. (canceled)

5. The method of claim 1, wherein the polypeptide of interest comprises a non-natural polypeptide or a theoretical non-natural polypeptide.

6. The method of claim 5, wherein the polypeptide comprises a polyepitope polypeptide comprising a tandem array of epitopes of interest.

7. A method for designing a candidate polyepitope polypeptide comprising a tandem array of epitopes for expression in a suitable host, said method comprising,
identifying the relative hydrophobicity of each of said epitopes,
dividing said epitopes on the basis of said identified hydrophobicities into groups of substantially equivalent numbers, said groups comprising at least a first group of epitopes of most relative hydrophobicity and a second group of epitopes of least relative hydrophobicity, and
arranging epitopes from said first and second groups in a substantially alternating manner so as to generate said candidate polyepitope polypeptide with reduced amplitude in hydrophobicity or length of a hydrophobic region.

8. A method for designing a candidate polyepitope polypeptide comprising a tandem array of epitopes for expression in a suitable host, said method comprising,
identifying the relative hydrophobicity of each of said epitopes,
dividing said epitopes on the basis of said identified hydrophobicities into three groups of substantially equivalent numbers, said groups comprising a first group of epitopes of most hydrophobicity, a second group of epitopes of intermediate relative hydrophobicity, and a third group of epitopes of least relative hydrophobicity,
arranging epitopes from said first, second and third groups into triplets containing an epitope from each group, and
arranging said triplets in a linked sequence so as to generate said candidate polyepitope polypeptide with reduced amplitude in hydrophobicity or length of a hydrophobic region.

9. A method for designing a candidate polyepitope polypeptide comprising a tandem array of epitopes for expression in a suitable host, said method comprising,
identifying the relative hydrophobicity of each of said epitopes,
dividing said epitopes on the basis of said identified hydrophobicities into four groups of substantially equivalent numbers, said groups comprising a first group of epitopes of most hydrophobicity, a second group of epitopes of lesser relative hydrophobicity, a third group of epitopes of even lesser relative hydrophobicity, and a fourth group of least relative hydrophobicity,
arranging epitopes from said first, second and third groups into quadruplets containing an epitope from each group, and
arranging said quadruplets in a linked sequence so as to generate said candidate polyepitope polypeptide with reduced amplitude in hydrophobicity or length of a hydrophobic region.

10. (canceled)

11. (canceled)

12. The method of any one of claims 7 to 9, wherein the epitopes comprising the polyepitope polypeptide are selected from epitopes of any one of the viruses of the group consisting of Epstein-Barr virus (EBV), hepatitis C virus (HCV), human immunodeficiency virus (HIV) and cytomegalovirus (CMV).

13. A method of expressing a polypeptide in a suitable host, said method comprising,
designing a polypeptide in accordance with the method of any one of claims 1, 7, 8, and 9,
introducing a polynucleotide encoding said polypeptide into said host, such that said host is capable of expressing said polypeptide, and
culturing said host under conditions suitable for expression of said polypeptide.

14. A polypeptide designed in accordance with the method of claim 1.

15. A polyepitope polypeptide designed in accordance with the method of claim 1.

16. A polyepitope polypeptide comprising N epitopes, wherein N is any integer, said polyepitope polypeptide having the formula
Triplet 1-Triplet2- . . . -Triplet N/3,
wherein each of said triplets comprises three linked epitopes selected by,
identifying and ranking the relative hydrophobicity of each of the N epitopes,
grouping the ranked N epitopes into three groups of substantially equivalent numbers, based upon the identified relative hydrophobicity of the N epitopes, to produce a first group comprising the epitopes of most relative hydrophobicity, a second group of epitopes of intermediate relative hydrophobicity, and a third group of epitopes of least relative hydrophobicity, and
selecting the epitopes for each of said triplets according to the following table:

| | Epitope 1 | Epitope 2 | Epitope 3 |
|---|---|---|---|
| Triplet 1 | Most | Most | Most |
| (N-terminal) | hydrophilic of | hydrophobic | hydrophilic of |
| | Group 2 | of Group 1 | Group 3 |
| Triplet 2 | 2^{nd} most | 2^{nd} most | 2^{nd} most |
| | hydrophilic of | hydrophobic of | hydrophilic |
| | Group 2 | Group 1 | of Group 3 |
| Triplet N/3 | Most | Most | Most |
| (C-terminal) | hydrophobic of | hydrophilic of | hydrophobic of |
| | Group 2 | Group 1 | Group 3 |

17. The polyepitope polypeptide of any one of claims 15, 16, 28, 29, and 30, wherein the epitopes are contiguous or spaced apart by intervening sequences which are substantially free of sequences which naturally flank said epitopes.

18. A polypeptide vaccine comprising a polyepitope polypeptide according to any one of claims 15, 16, 28, 29 and 30, and a pharmaceutically acceptable carrier or adjuvant.

19. A polyepitope polypeptide comprising an amino acid sequence substantially corresponding to an amino acid sequence selected from the group consisting of:
(SEQ ID NO: 1)
FLRGRAYGL-PYLFWLAAI-HRCQAIRKK-RRIYDLIEL-
VQPPQLTLQVGLCTLVAML-RLRAEAQVK-IEDPPFNSL-
YLLEMLWRL-GQGGSPTAMAVLLHEESM-IALYLQQNWWTL-
RAKFKQLL-SSCSSCPLSKI-TYGPVFMCLQAKWRLQTL-
RPPIFIRRL-VSFIEFVGW-YPLHEQHGM-
VEITPYKPTWCLGGLLTMV-EENLLDFVRF-TYSAGIVQI-
LLDFVRFGV-EGGVGWRHW,
(SEQ ID NO: 2)
FLRGRAYGL-PYLFWLAAI-HRCQAIRKK-RRIYDLIEL-
GLCTLVAMLRLRAEAQVK-IEDPPFNSL-TYSAGIVQI-
LLDFVRFMGV-EGGVGWRHWIALYLQQNWWTL-RAKFKQLL-
SSCSSCPLSKI-TYGPVFMCL-QAKWRLQTLRPPIFIRRL-
VSFIEFVGW-YPLHEQHGM-VEITPYKPTW-
CLGGLLTMVEENLLDFVRF-YLLEMLWRL-GQGGSPTAM-
AVLLHEESM-VQPPQLTLQV,
(SEQ ID NO: 3)
SSCSSCPLSKI-HRCQAIRKK-CLGGLLTMV-LTAGFLIFL-
RLRAEAQVK-IEDPFNSL-LLSAWILTA-RRRWRRLTV-
PYLFWLAAI-YLLEMLWRL-GQGGSPTAM-VMSNTLLSAW-
ALLVLYSFA-RAKFKQLL-IALYLQQNW-TYGPVFMCL-
QAKWRLQTL-YLQQNWWTL-YPLHEQHGM-CPLSKILL,
(SEQ ID NO: 4)
IPIVAIVALV-RLRPGGKKK-ILKEPVHGV-PLVKLWYQL-
RPGGKKKYKL-KYKLKHIVW-TWETWWTEYW-EIKDTKEAL-
KRWIILGLNK-KLWVTVYYGV-KIEELRQHL-MTNNPPIPV-
VTLWQRPLV-WASRELERF-LLWKGEGAV-YTAFTIPSI-
IYQEPFKNLK-SLYNTVATL-AIIRILQQL-AIFQSSMTK-
VIYQYMDDL-LVGPTPVNI-TPQDLNTML-YLAWVPAHK-
ALVEICTEM-TLNAWVKVV,
and
(SEQ ID NO: 5)
LLFNILGGWV-KTSERSQPR-FLLLADARV-LLFLLLADA-
RLGVRATRK-GVAGALVAFK-LPGCSFSIF-RMYVGGVEHR-
VAGALVAFK-DLMGYIPLV-LIFCHSKKK-ILAGYGAGV-
HMWNFISGI-QLFTFSPRR-VGIYLLPNR-FWAKHMWNF-
YLVTRHADV-LSAFSLHSY-WMNRLIAFA-YLLPRRGPRL-
YLVAYQATV-RLIVFPDLGV-TLGFGAYMSK-IPFYGKAI-
VLVGGVLAA-CTCGSSDLY

20. The polyepitope polypeptide of claim 19, wherein the polyepitope polypeptide comprises an amino acid sequence substantially corresponding to:
(SEQ ID NO: 1)
FLRGRAYGL-PYLFWLAAI-HRCQAIRKK-RRIYDLEIL-
VQPPQLTLQV-GLCTLVAML-RLRAEAQVK-IEDPPFNSL-
YLLEMLWRL-GQGGSPTAM-AVLLHEESM-IALYLQQNWWTL-
RAKFKQLL-SSCSSCPLSKI-TYGPVFMCL-QAKWRLQTL-
RPPIFIRRL-VSFIEFVGW-YPLHEQHGM-VEITPYKPTW-
CLGGLLTMV-EENLLDFVRF-TYSAGIVQI-LLDFVRFMGV-
EGGVGWRHW.

21. The polyepitope polypeptide of claim 19, wherein the polyepitope polypeptide comprises an amino acid sequence substantially corresponding to:
(SEQ ID NO: 2)
FLRGRAYGL-PYLFWLAAI-HRCQAIRKK-RRIYDLIEL-
GLCTLVAML-RLRAEAQVK-IEDPPFNSL-TYSAGIVQI-
LLDFVRFMGV-EGGVGWRHW-IALYLQQNWWTL-RAKFKQLL-
SSCSSCPLSKI-TYGPVFMCL-QAKWRLQTL-RPPIFIRRL-
VSFIEFVGW-YPLHEQHGM-VEITPYKPTW-CLGGLLTMV-
EENLLDFVRF-YLLEMLWRL-GQGGSPTAM-AVLLHEESM-
VQPPQLTLQV.

22. The polyepitope polypeptide of claim 19, wherein the polyepitope polypeptide comprises an amino acid sequence substantially corresponding to:
(SEQ ID NO:3)
SSCSSCPLSKI-HRCQAIRKK-CLGGLLTMV-LTAGFLIFL-
RLRAEAQVK-IEDPPFNSL-LLSAWILTA-RRRWRRLTV-
PYLFWLAAI-YLLEMLWRL-GQGGSPTAM-VMSNTLLSAW-
ALLVYLSFA-RAKFKQLL-IALYLQQNW-TYGPVFMCL-
QAKWRLQTL-YLQQNWWTL-YPLHEQHGM-CPLSKILL.

23. The polyepitope polypeptide of claim 19, wherein the polyepitope polypeptide comprises an amino acid sequence substantially corresponding to:
(SEQ ID NO: 4)
IPIVAIVALV-RLRPGGKKK-ILKEPVHGV-PLVKLWYQL-
RPGGKKKYKL-KYKLKHIVW-TWETWWTEYW-EIKDTKEAL-
KRWIILGLNK-KLWVTVYYG-KIEELRQHL-MTNNPPIPV-
VTLWQRPLV-WASRELERF-LLWKGEGAV-YTAFTIPSI-
IYQEPFKNLK-SLYNTVATL-AIIRILQQL-AIFQSSMTK-
VIYQYMDDL-LVGPTPVNI-TPQDLNTML-YLAWVPAHK-
ALVEICTEM-TLNAWVKW.

24. The polyepitope polypeptide of claim 19, wherein the polyepitope polypeptide comprises an amino acid sequence substantially corresponding to:
(SEQ ID NO: 5)
LLFNILGGWV-KTSERSQPR-FLLLADARV-LLFLLLADA-
RLGVRATRK-GVAGALVAFK-LPGCSFSIF-RMYVGGVEHR-
VAGALVAFK-DLMGYWLV-LIIFCHSKKK-ILAGYGAGV-
HMWNFISGI-QLFTFSPRR-VGIYLLPNR-FWAKHMWNF-
YLVTRHADV-LSAFSLHSY-WMNRLIAFA-YLLPRRGPRL-
YLVAYQATV-RLIVFPDLGV-TLGFGAYMSK-IPFYGKAI-
VLVGGVLAA-CTCGSSDLY.

25. A polypeptide vaccine comprising a polyepitope polypeptide according to claim 19 and a pharmaceutically acceptable carrier or adjuvant.

26. A viral or DNA vaccine comprising a polynucleotide encoding a polypeptide designed in accordance with the method of any one of claims 1, 7, 8, and 9 and a pharmaceutically acceptable carrier or adjuvant.

27. A viral or DNA vaccine comprising a polynucleotide encoding a polypeptide according to claim 19 and a pharmaceutically acceptable carrier or adjuvant.

28. A polyepitope polypeptide designed in accordance with the method of claim 7.

29. A polyepitope polypeptide designed in accordance with the method of claim 8.

30. A polyepitope polypeptide designed in accordance with the method of claim 9.


SEQUENCE LISTING (102 sequences)

SEQ ID NO: 1 - protein, 233 aa, epstein-barr virus (EBV)
FLRGRAYGLPYLFWLAAIHRCQAIRKKRRIYDLIELVQPPQLTLQVGLCTLVAMLRLRAEAQVKIEDPPFNSLYLLEMLWRLGQGGSPTAMAVLLHEESMIALYLQQNWWTLRAKFKQLLSSCSSCPLSKITYGPVFMCLQAKWRLQTLRPPIFIRRLVSFIEFVGWYPLHEQHGMVEITPYKPTWCLGGLLTMVEENLLDFVRFTYSAGIVQILLDFVRFMGVEGGVGWRHW

SEQ ID NO: 2 - protein, 233 aa, epstein-barr virus (EBV)
FLRGRAYGLPYLFWLAAIHRCQAIRKKRRIYDLIELGLCTLVAMLRLRAEAQVKIEDPPFNSLTYSAGIVQILLDFVRFMGVEGGVGWRHWIALYLQQNWWTLRAKFKQLLSSCSSCPLSKITYGPVFMCLQAKWRLQTLRPPIFIRRLVSFIEFVGWYPLHEQHGMVEITPYKPTWCLGGLLTMVEENLLDFVRFYLLEMLWRLGQGGSPTAMAVLLHEESMVQPPQLTLQV

SEQ ID NO: 3 - protein, 181 aa, epstein-barr virus (EBV)
SSCSSCPLSKIHRCQAIRKKCLGGLLTMVLTAGFLIFLRLRAEAQVKIEDPPFNSLLLSAWILTARRRWRRLTVPYLFWLAAIYLLEMLWRLGQGGSPTAMVMSNTLLSAWALLVLYSFARAKFKQLLIALYLQQNWTYGPVFMCLQAKWRLQTLYLQQNWWTLYPLHEQHGMCPLSKILL

SEQ ID NO: 4 - protein, 240 aa, Human immunodeficiency virus
IPIVAIVALVRLRPGGKKKILKEPVHGVPLVKLWYQLRPGGKKKYKLKYKLKHIVWTWETWWTEYWEIKDTKEALKRWIILGLNKKLWVTVYYGVKIEELRQHLMTNNPPIPVVTLWQRPLVWASRELERFLLWKGEGAVYTAFTIPSIIYQEPFKNLKSLYNTVATLAIIRILQQLAIFQSSMTKVIYQYMDDLLVGPTPVNITPQDLNTMLYLAWVPAHKALVEICTEMTLNAWVKVV

SEQ ID NO: 5 - protein, 239 aa, Hepatitis C virus
LLFNILGGWVKTSERSQPRFLLLADARVLLFLLLADARLGVRATRKGVAGALVAFKLPGCSFSIFRMYVGGVEHRVAGALVAFKDLMGYIPLVLIFCHSKKKILAGYGAGVHMWNFISGIQLFTFSPRRVGIYLLPNRFWAKHMWNFYLVTRHADVLSAFSLHSYWMNRLIAFAYLLPRRGPRLYLVAYQATVRLIVFPDLGVTLGFGAYMSKIPFYGKAIVLVGGVLAACTCGSSDLY

SEQ ID NO: 6 - protein, 9 aa, epstein-barr virus (EBV)
CLGGLLTMV

SEQ ID NO: 7 - protein, 9 aa, epstein-barr virus (EBV)
GLCTLVAML

SEQ ID NO: 8 - protein, 10 aa, epstein-barr virus (EBV)
LLDFVRFMGV

SEQ ID NO: 9 - protein, 9 aa, epstein-barr virus (EBV)
YLLEMLWRL

SEQ ID NO: 10 - protein, 9 aa, epstein-barr virus (EBV)
YLQQNWWTL

SEQ ID NO: 11 - protein, 9 aa, epstein-barr virus (EBV)
RLRAEAQVK

SEQ ID NO: 12 - protein, 11 aa, epstein-barr virus (EBV)
SSCSSCPLSKI

SEQ ID NO: 13 - protein, 9 aa, epstein-barr virus (EBV)
PYLFWLAAI

SEQ ID NO: 14 - protein, 9 aa, epstein-barr virus (EBV)
TYGPVFMCL

SEQ ID NO: 15 - protein, 9 aa, epstein-barr virus (EBV)
TYSAGIVQI

SEQ ID NO: 16 - protein, 9 aa, epstein-barr virus (EBV)
RPPIFIRRL

SEQ ID NO: 17 - protein, 9 aa, epstein-barr virus (EBV)
FLRGRAYGL

SEQ ID NO: 18 - protein, 9 aa, epstein-barr virus (EBV)
QAKWRLQTL

SEQ ID NO: 19 - protein, 8 aa, epstein-barr virus (EBV)
RAKFKQLL

SEQ ID NO: 20 - protein, 9 aa, epstein-barr virus (EBV)
HRCQAIRKK

SEQ ID NO: 21 - protein, 9 aa, epstein-barr virus (EBV)
RRIYDLIEL

SEQ ID NO: 22 - protein, 9 aa, epstein-barr virus (EBV)
AVLLHEESM

SEQ ID NO: 23 - protein, 9 aa, epstein-barr virus (EBV)
YPLHEQHGM

SEQ ID NO: 24 - protein, 10 aa, epstein-barr virus (EBV)
EENLLDFVRF

SEQ ID NO: 25 - protein, 9 aa, epstein-barr virus (EBV)
EGGVGWRHW

SEQ ID NO: 26 - protein, 10 aa, epstein-barr virus (EBV)
VEITPYKPTW

SEQ ID NO: 27 - protein, 10 aa, epstein-barr virus (EBV)
VQPPQLTLQV

SEQ ID NO: 28 - protein, 9 aa, epstein-barr virus (EBV)
IALYLQQNW

SEQ ID NO: 29 - protein, 9 aa, epstein-barr virus (EBV)
VSFIEFVGW

SEQ ID NO: 30 - protein, 9 aa, epstein-barr virus (EBV)
IEDPPFNSL

SEQ ID NO: 31 - protein, 9 aa, epstein-barr virus (EBV)
GQGGSPTAM

SEQ ID NO: 32 - DNA, 30 bp, epstein-barr virus (EBV)
agggatcccatggaagatcctggttttttc

SEQ ID NO: 33 - DNA, 31 bp, epstein-barr virus (EBV)
tctagaggtcgacacctgtcgttgtattggg

SEQ ID NO: 34 - protein, 12 aa, epstein-barr virus (EBV)
IALYLQQNWWTL

SEQ ID NO: 35 - protein, 6 aa, Hexa-histidine tag
HHHHHH

SEQ ID NO: 36 - protein, 10 aa, epstein-barr virus (EBV)
VMSNTLLSAW

SEQ ID NO: 37 - protein, 9 aa, epstein-barr virus (EBV)
LLSAWILTA

SEQ ID NO: 38 - protein, 9 aa, epstein-barr virus (EBV)
LTAGFLIFL

SEQ ID NO: 39 - protein, 8 aa, epstein-barr virus (EBV)
CPLSKILL

SEQ ID NO: 40 - protein, 9 aa, epstein-barr virus (EBV)
RRRWRRLTV

SEQ ID NO: 41 - protein, 9 aa, epstein-barr virus (EBV)
ALLVLYSFA

SEQ ID NO: 42 - protein, 9 aa, Human immunodeficiency virus
RLRPGGKKK

SEQ ID NO: 43 - protein, 9 aa, Human immunodeficiency virus
KYKLKHIVW

SEQ ID NO: 44 - protein, 9 aa, Human immunodeficiency virus
WASRELERF

SEQ ID NO: 45 - protein, 10 aa, Human immunodeficiency virus
RPGGKKKYKL

SEQ ID NO: 46 - protein, 9 aa, Human immunodeficiency virus
SLYNTVATL

SEQ ID NO: 47 - protein, 9 aa, Human immunodeficiency virus
EIKDTKEAL

SEQ ID NO: 48 - protein, 9 aa, Human immunodeficiency virus
TLNAWVKVV

SEQ ID NO: 49 - protein, 9 aa, Human immunodeficiency virus
TPQDLNTML

SEQ ID NO: 50 - protein, 9 aa, Human immunodeficiency virus
MTNNPPIPV

SEQ ID NO: 51 - protein, 10 aa, Human immunodeficiency virus
KRWIILGLNK

SEQ ID NO: 52 - protein, 9 aa, Human immunodeficiency virus
VTLWQRPLV

SEQ ID NO: 53 - protein, 9 aa, Human immunodeficiency virus
LVGPTPVNI

SEQ ID NO: 54 - protein, 9 aa, Human immunodeficiency virus
ALVEICTEM

SEQ ID NO: 55 - protein, 9 aa, Human immunodeficiency virus
YTAFTIPSI

SEQ ID NO: 56 - protein, 9 aa, Human immunodeficiency virus
AIFQSSMTK

SEQ ID NO: 57 - protein, 9 aa, Human immunodeficiency virus
VIYQYMDDL

SEQ ID NO: 58 - protein, 9 aa, Human immunodeficiency virus
KIEELRQHL

SEQ ID NO: 59 - protein, 9 aa, Human immunodeficiency virus
ILKEPVHGV

SEQ ID NO: 60 - protein, 10 aa, Human immunodeficiency virus
IYQEPFKNLK

SEQ ID NO: 61 - protein, 10 aa, Human immunodeficiency virus
TWETWWTEYW

SEQ ID NO: 62 - protein, 9 aa, Human immunodeficiency virus
PLVKLWYQL

SEQ ID NO: 63 - protein, 9 aa, Human immunodeficiency virus
YLAWVPAHK

SEQ ID NO: 64 - protein, 9 aa, Human immunodeficiency virus
LLWKGEGAV

SEQ ID NO: 65 - protein, 9 aa, Human immunodeficiency virus
AIIRILQQL

SEQ ID NO: 66 - protein, 10 aa, Human immunodeficiency virus
IPIVAIVALV

SEQ ID NO: 67 - protein, 10 aa, Human immunodeficiency virus
KLWVTVYYGV

SEQ ID NO: 68 - protein, 9 aa, Hepatitis C virus
CTCGSSDLY

SEQ ID NO: 69 - protein, 9 aa, Hepatitis C virus
FLLLADARV

SEQ ID NO: 70 - protein, 9 aa, Hepatitis C virus
YLVAYQATV

SEQ ID NO: 71 - protein, 10 aa, Hepatitis C virus
RLIVFPDLGV

SEQ ID NO: 72 - protein, 9 aa, Hepatitis C virus
DLMGYIPLV

SEQ ID NO: 73 - protein, 9 aa, Hepatitis C virus
WMNRLIAFA

SEQ ID NO: 74 - protein, 9 aa, Hepatitis C virus
VLVGGVLAA

SEQ ID NO: 75 - protein, 9 aa, Hepatitis C virus
HMWNFISGI

SEQ ID NO: 76 - protein, 9 aa, Hepatitis C virus
ILAGYGAGV

SEQ ID NO: 77 - protein, 10 aa, Hepatitis C virus
YLLPRRGPRL

SEQ ID NO: 78 - protein, 9 aa, Hepatitis C virus
LLFLLLADA

SEQ ID NO: 79 - protein, 9 aa, Hepatitis C virus
YLVTRHADV

SEQ ID NO: 80 - protein, 9 aa, Hepatitis C virus
KTSERSQPR

SEQ ID NO: 81 - protein, 9 aa, Hepatitis C virus
RLGVRATRK

SEQ ID NO: 82 - protein, 9 aa, Hepatitis C virus
QLFTFSPRR

SEQ ID NO: 83 - protein, 10 aa, Hepatitis C virus
RMYVGGVEHR

SEQ ID NO: 84 - protein, 9 aa, Hepatitis C virus
LIFCHSKKK

SEQ ID NO: 85 - protein, 10 aa, Hepatitis C virus
GVAGALVAFK

SEQ ID NO: 86 - protein, 9 aa, Hepatitis C virus
VAGALVAFK

SEQ ID NO: 87 - protein, 10 aa, Hepatitis C virus
TLGFGAYMSK

SEQ ID NO: 88 - protein, 9 aa, Hepatitis C virus
LPGCSFSIF

SEQ ID NO: 89 - protein, 9 aa, Hepatitis C virus
LSAFSLHSY

SEQ ID NO: 90 - protein, 9 aa, Hepatitis C virus
FWAKHMWNF

SEQ ID NO: 91 - protein, 9 aa, Hepatitis C virus
VGIYLLPNR

SEQ ID NO: 92 - protein, 10 aa, Hepatitis C virus
LLFNILGGWV

SEQ ID NO: 93 - protein, 8 aa, Hepatitis C virus
IPFYGKAI

SEQ ID NO: 94 - protein, 242 aa, epstein-barr virus (EBV)
MVDFLRGRAYGLPYLFWLAAIHRCQAIRKKRRIYDLIELVQPPQLTLQVGLCTLVAMLRLRAEAQVKIEDPPFNSLYLLEMLWRLGQGGSPTAMAVLLHEESMIALYLQQNWWTLRAKFKQLLSSCSSCPLSKITYGPVFMCLQAKWRLQTLRPPIFIRRLVSFIEFVGWYPLHEQHGMVEITPYKPTWCLGGLLTMVEENLLDFVRFTYSAGIVQILLDFVRFMGVEGGVGWRHWHHHHHH

SEQ ID NO: 95 - protein, 242 aa, epstein-barr virus (EBV)
MVDFLRGRAYGLPYLFWLAAIHRCQAIRKKRRIYDLIELGLCTLVAMLRLRAEAQVKIEDPPFNSLTYSAGIVQILLDFVRFMGVEGGVGWRHWIALYLQQNWWTLRAKFKQLLSSCSSCPLSKITYGPVFMCLQAKWRLQTLRPPIFIRRLVSFIEFVGWYPLHEQHGMVEITPYKPTWCLGGLLTMVEENLLDFVRFYLLEMLWRLGQGGSPTAMAVLLHEESMVQPPQLTLQVHHHHHH

SEQ ID NO: 96 - protein, 669 aa, epstein-barr virus (EBV)
MEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGVDFLRGRAYGLPYLFWLAAIHRCQAIRKKRRIYDLIELVQPPQLTLQVGLCTLVAMLRLRAEAQVKIEDPPFNSLYLLEMLWRLGQGGSPTAMAVLLHEESMIALYLQQNWWTLRAKFKQLLSSCSSCPLSKITYGPVFMCLQAKWRLQTLRPPIFIRRLVSFIEFVGWYPLHEQHGMVEITPYKPTWCLGGLLTMVEENLLDFVRFTYSAGIVQILLDFVRFMGVEGGVGWRHWHHHHHH

SEQ ID NO: 97 - protein, 669 aa, epstein-barr virus (EBV)
MEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGVDFLRGRAYGLPYLFWLAAIHRCQAIRKKRRIYDLIELGLCTLVAMLRLRAEAQVKIEDPPFNSLTYSAGIVQILLDFVRFMGVEGGVGWRHWIALYLQQNWWTLRAKFKQLLSSCSSCPLSKITYGPVFMCLQAKWRLQTLRPPIFIRRLVSFIEFVGWYPLHEQHGMVEITPYKPTWCLGGLLTMVEENLLDFVRFYLLEMLWRLGQGGSPTAMAVLLHEESMVQPPQLTLQVHHHHHH

SEQ ID NO: 98 - protein, 187 aa, epstein-barr virus (EBV)
SSCSSCPLSKIHRCQAIRKKCLGGLLTMVLTAGFLIFLRLRAEAQVKIEDPPFNSLLLSAWILTARRRWRRLTVPYLFWLAAIYLLEMLWRLGQGGSPTAMVMSNTLLSAWALLVLYSFARAKFKQLLIALYLQQNWTYGPVFMCLQAKWRLQTLYLQQNWWTLYPLHEQHGMCPLSKILLHHHHHH

SEQ ID NO: 99 - protein, 246 aa, Human immunodeficiency virus
IPIVAIVALVRLRPGGKKKILKEPVHGVPLVKLWYQLRPGGKKKYKLKYKLKHIVWTWETWWTEYWEIKDTKEALKRWIILGLNKKLWVTVYYGVKIEELRQHLMTNNPPIPVVTLWQRPLVWASRELERFLLWKGEGAVYTAFTIPSIIYQEPFKNLKSLYNTVATLAIIRILQQLAIFQSSMTKVIYQYMDDLLVGPTPVNITPQDLNTMLYLAWVPAHKALVEICTEMTLNAWVKVVHHHHHH

SEQ ID NO: 100 - protein, 246 aa, Human immunodeficiency virus
ILKEPVHGVKRWIILGLNKTLNAWVKVVKIEELRQHLYTAFTIPSIWASRELERFPLVKLWYQLLVGPTPVNIIPIVAIVALVSLYNTVATLMTNNPPIPVLLWKGEGAVTWETWWTEYWAIFQSSMTKRLRPGGKKKALVEICTEMKLWVTVYYGVYLAWVPAHKAIIRILQQLEIKDTKEALVTLWQRPLVIYQEPFKNLKVIYQYMDDLTPQDLNTMLRPGGKKKYKLKYKLKHIVWHHHHHH

SEQ ID NO: 101 - protein, 245 aa, Hepatitis C virus
LLFNILGGWVKTSERSQPRFLLLADARVLLFLLLADARLGVRATRKGVAGALVAFKLPGCSFSIFRMYVGGVEHRVAGALVAFKDLMGYIPLVLIFCHSKKKILAGYGAGVHMWNFISGIQLFTFSPRRVGIYLLPNRFWAKHMWNFYLVTRHADVLSAFSLHSYWMNRLIAFAYLLPRRGPRLYLVAYQATVRLIVFPDLGVTLGFGAYMSKIPFYGKAIVLVGGVLAACTCGSSDLYHHHHHH

SEQ ID NO: 102 - protein, 245 aa, Hepatitis C virus
YLVTRHADVWMNRLIAFACTCGSSDLYVGIYLLPNRILAGYGAGVLIFCHSKKKHMWNFISGIFLLLADARVLLFLLLADALSAFSLHSYGVAGALVAFKKTSERSQPRRLGVRATRKDLMGYIPLVVLVGGVLAALLFNILGGWVYLVAYQATVYLLPRRGPRLFWAKHMWNFQLFTFSPRRLPGCSFSIFIPFYGKAIRMYVGGVEHRTLGFGAYMSKRLIVFPDLGVVAGALVAFKHHHHHH